(19) United States
(12) Patent Application Publication
(10) Pub. No.: US 2004/0126437 A1
(43) Pub. Date: Jul. 1, 2004

(54) Typing natural products

(76) Inventor: Maria Cristina Marcucci Ribeiro, Campinas (BR)

Correspondence Address: SHAPIRO COHEN, P.O. BOX 3440, STATION D, OTTAWA, ON, K1P6P1, CA

(21) Appl. No.: 10/433,422

(22) PCT Filed: Dec. 26, 2001
(86) PCT No.: PCT/BR01/00159

(30) Foreign Application Priority Data
Dec. 22, 2000 (BR) PI 0006272-3

(51) Int. Cl. A61K 35/78

(52) U.S. Cl. 424/725

(57) ABSTRACT

The present invention refers to achemical process for separation of different kinds of substances which are present in Brazilian propolis, by means of HPLC and GC-MS combined techniques. This provided a process for a HPLC separation which is able to identify the majority components of the propolis sample by using a suitable analysis software for this end.

The present invention refers to a chemical process for separate different kinds of substances from the Brazilian propolis, by means of HPLC and gas chromatography combined to mass spectrometry. Only these two techniques, are able to permit the identification and quantification, with high precision, of the different compounds from the Brazilian propolis. A separation process by means of HPLC for identify major components of propolis samples has been also provided. The gas chromatography allied to to the mass spectrometry play an excelent complementary function in view of the HPLC method of the invention, since samples having the same output profile in the HPLC method produce different results via gas chromatography. Based on the results of several sample analysis the present invention is able to determine methodologies of chemical analysis related to the Brazilian propolis. The HPLC technique and the quantification of the identified compounds has permitted the typing of the Brazilian propolis based on the chemical marker, the 3,5-diprenyl4-hydroxycinnamic acid. The process of the invention also includes a data input into a electronic schedule for the quantification of the HPLC identified substances and typing is determined based on the amounts of each component from the Brazilian propolis chemical composition.

### BACKGROUND OF THE INVENTION

Propolis from tropical reigions, especially in Brazil, have been focused as one of the World economical point of interest on last years, and Japan is the major world acquirer of the Brazilian propolis. This economical point has caused an increased the scientific interest, particularly referring to Japan scientists, for studying the Brazilian propolis and this also increased the scientific publication related to the Brazilian propolis in the World.

Propolis source in seasoned zones

The first reports related to propolis analysis based on chemical evidences raised on the ′70 decade when Lavie (Lavie, P., Proc. XXV Int. Beekeeping Congr., Grenoble, Bucharest, Apimondia, 229-233, 1975) in French and Popravko (Popravko, S. A.: in “A remarkable Hive Product: Propolis, Apimondia, Bucharest, 15-18, 1978) in Russia identified the presence of flavonoids in propolis and these scientists compared this with exsudates from Populus and Betula species. Many other publications have been issued, presently, it is recognized that the principal source of propolis in seasoned regions are buds and exsudates of Populus and its hibrids variations. A skilled person in this art can confirm the above dicussion by consulting in Europe (Tamas et al., Stud. Cercet.Biochim., 22, 207-213, 1979; Popravko et Sokolov, Pchelovodstvo 2, 28-29, 1980; Nagy et al., Stud.Org.Chem., 23, 223-232, 1986; Greenaway et al., Proc. R.Soc.London B, 232, 249-272, 1987; Bankova et Kuleva, Animal Sci., 26, 94-98, 1989;), in North America (García-Viguera et al., Z.Naturforshung, 48C, 731-735, 1993) and non tropical regions of Asia (Bankova et al., Apidologie, 23, 79-85, 1992, Chi et al. Z. Yaoxue Zazhi, 31, 264-266, 1996). Even in New Zealand, men introduced Populus species, are the bee preferred propolis source (Markham et al.Phytochem., 42, 205-211, 1996). In Russia, particularly in the northern region the Betula species (Betula verrucosa) are also the bee preferred propolis source (Popravko e Sokolov, Pchelovodstvo, 2, 28-29, 1980).

Propolis source from the above mentioned regions has a very similar chemical composition and the principal components are flavonoids, aglicones, aromatic acids and their esters (Marcucci, Apidologie 26, 83-99, 1995).

The formula below is the phenolic benzopyran derivatives chemical structure wich have been identified by Markonius, 1995, 1999.

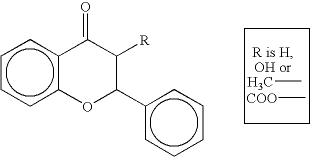

With regard to the European propolis, many patents have been granted, special mention to the Swedians by reporting the presence of phenolic bezopyran derivatives having antiviral & antibaterial and immunoestimulation activity (Markonius, U.S. Pat. No. 5,449,794, 1995, U.S. Pat. No. 5,861,430, 1999).

Propolis source in tropical zones

There are no Populus and Betula species in tropical zones, therefore, other plants are the bee and the chemical composition of propolis sample of said regions is different from the seasoned regions (Bankova et al., Apidologie, 31, 3-15, 2000). Investigations related to said resin in tropical regions has revealed that flavonoid are the principal component of the propolis samples, similarly to the Eurpean ones, even having different plant origin. Wollenweber & Buchmann (Z.Naturforschung., 52C, 530-535, 1997), for exemple, identified some aglicone flavonoids in samples from exsudates of Ambrosia deltoidea in the Sonora desert: 3,5,7-trihydroxy-3,8-dimethoxyflavone, xanthomicrol, hispiduline, sideritiflavone, kaempferol, quercetine, ramnazine and other minor components. Miricetine 3,7,4′,5′-tetramethyl ether and quercetine 3,7,3′-trimethyl ether have been insulated from Tunisian propolis which has extracted from leaf exsudates of some Citrus species (Martos et al. J.Agric.Food Chem., 54, 2824-2829, 1997).

In the Brazilian samples, kaempferide, 5,6,7-trihydroxy-3,4′-dihydroxyflavone, aromadendrine-4-methyl ether, kaempferol and apigenine, were the some of few identified flavonoids (Boudourova-Krasteva. et al., Z.Naturforschung, 52C, 676-679, 1997), and more the pinobanksine and one kaempferol derivative (Marcucci et al. Z.Naturforschung., 55C, 76-81, 2000). Besides, in these samples, other new compounds having remarkable biological activity have been identified (Bankova et al. Apidologie 31, 3-15, 2000). Another class of phenolics, the prenyl p-coumaric acids have been found in large amount in Brazilian sample (Aga et al., Biosci.Biotech.Biochem., 58, 945-946,1994; Boudourova-Krasteva et al. Z.Naturforschung, 52C, 676-679, 1997). 3-prenyl4-hydroxycinnamic acid (PHCA), 9-E- e 9-Z-2,2-dimethyl-6-carboxiethenyl-8-prenyl-2H-1-benzopyran (DCBEN) (Marcucci, Post-Doc. Report Ext. Fapesp, Protocol Registry No. 94/03686-8, 1996), 3,5-diprenyl-4-hydroxycinnamic acid (DHCA) and 6-propenoic-2,2-dimethyl-8-prenyl-2H-1-benzopyran (DPB). The above reported compounds, PHCA, DCBEN, DHCA and DPB, 3-methoxy4-hydroxycinnamic aldehyde (G2), 2-[1-hidroximethyl]-vinyl-6-acetyl-5-hydroxycumaran (I) (From Baccharis truncata) and the unknowns: H, K, L1, L2, M and N have been insulated by the author during her Post-Doctorade (Fapesp Protocol Registry No. 94/3686-8).

Some of the above compounds has antimicrobial and antitumoral activity (Aga et al., Biosci.Biotech.Biochem., 58, 945-946, 1994, Banskota et al., J.Nat.Prod., 61, 896-900, 1998). Flavonoids are absent of these samples (Bankova et al. Z.Naturforschung, 50C, 167-172, 1995).

The chemical structure of some of the above mentioned compounds are shown as follows:

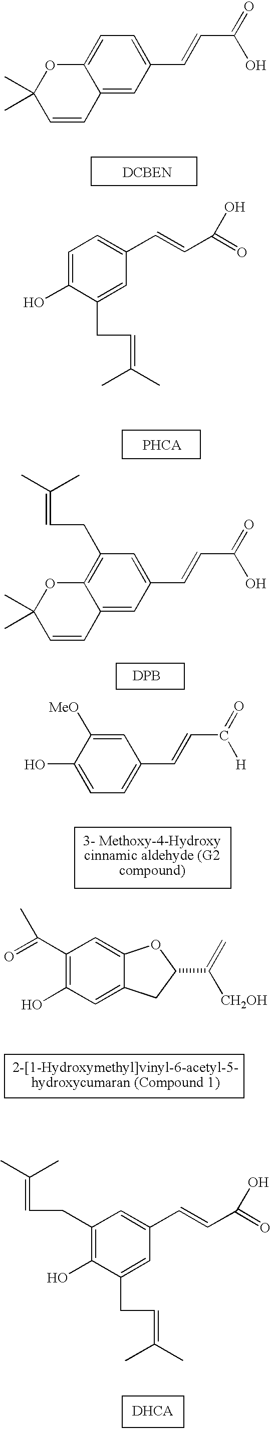

Th identified compounds from Brazilian propolis

The insulated compounds DCBEN, DHCA, E and mixture of E+DPB have their antibacterial properties tested in cultures of E.coli, Pseudomonas aeruginosa, Staphylococcus aureus and Streptococcus faecalis. Inhibition halos (in mm) and the obtained data recorded as in FIG. 1. S.aureus and S.faecalis were the most affected by the tested compounds, just varying the diameter of the inhibition halos; the mixture E+DPB has show more toxic effect. With respect to E.coli, only DHCA did not present inhibition activity, but and in P.aeruginosa, the mixture E+DPB has shown toxic effect.

FIG. 2 are recorded ID₅₀ data related to trypomastigotes cultures (one of the growing stages of Trypanosoma cruzi (T.cruzi)) also to the compounds DCBEN, DHCA, E and mixture E+DPB and the effects of said compounds extracted from propolis trypomastigotes of T.cruzi (in mg/mL). The results shows differences between the ID₅₀ to all analysed compounds having values ranging from 0.932-1.696 mg/mL. The same result for Violet Crystal is 0.205 mg/mL, as previously mentioned. The results for insulated compounds are greater than the Violet Crystal.

Paulino et al. (Naunyn Schiedebergs Arch. Pharmacol., 360, 331-336, 1999) have demonstrated the relation between the presence of the identified compounds in propolis sample by HPLC, and the analgesic activity of the exsudates. The same authors report that preparations from insulated cavy trachea (ICT) previously contracted with hystamine (1 μM) and submitted to a concentration-relaxation response curve incubated with propolis extract containing the DHCA compound, shows CE₅₀ 282 (195-408) μg/mL (dosage inducing 50% of relaxation) and Emax 1.30 (±0.09)g de tension. The compounds PHCA, DCBEN and I, induced relaxation similarly to the propolis extract. The compound PHCA: CE₅₀ 3,26 (0.59-18,0) and Emax 1.258±84 g tension. The compound DCBEN: CE₅₀ 0.25 (0.09-1.30) μM and Emax 0.80±0.05 g, and the compound I: CE₅₀ 0.44 (0.13-1.51) mM and Emax 1.06±0.09 g. These results have demonstrated that the tested propolis presents relaxation response in the smooth muscle of the ICTand the compounds PHCA, DCBEN e I, can contribute in part at least for this effect (Marcucci et al., J.Ethnopharmacol., 74,105-112 (2001).

Antibacterial activity of the compounds K, G₁, G₂ e L₂ has been evaluated in cultures of E.coli, Pseudomonas aeruginosa, Staphylococcus aureus and Streptococcus faecalis. Inhibition halos have been measured (mm) and the data recorded as in FIG. 1. S.aureus, S.faecalis e E.coli, are sensitive to the tested compounds, just varying the halos, the compound G₂ has the most toxity in cultures of S.aureus e S.faecalis. None of the tested compounds had effect in P.aeruginosa.

Other phenolic substances than the acids previous reported have been found: derivatives of cafeolquinic acid having immuno ondulatory and hepatoprotective action (chlorogenic acid: 4-caffeoylquinic acid, 5-caffeoylquinic acid, 3,5-dicaffeoylquinic acid e 4,5-dicaffeoylquinic acid and its methyl esther, acid: 3,4-dicaffeoylquinic e its methyl esther) (Tatefuji et al., Biol.Pharm.Bull., 19, 966-970, 1996; Basnet et al., Z.Naturforschung., 52C, 828-833, 1996).; lignan, 2,3-dihydro-2-(4-hydroxy-3-methoxyphenyl)-5 (3-acetoxi-E-propenyl)-3-acetoximethyl-benzofuran (Bankova et al., Z.Naturforschung., 51B, 735-737, 1996) free radical supressor: 3-[b 4-hydroxy-3-(3-oxobut-1-enyl)-phenyl]acrylic acid (Basnet et al., Z.Naturforschung., 52C, 828-833, 1996).

The diterpenes are other class of important compounds which are present in Brazilian propolis. Four compounds are diterpenes according to their mass spectra. A detailed analysis of the mass spectra and MNR has revealed that the compounds are diterpenic acids having a labdan skeleton and by compairson with the literature data, the compounds are identified as follows: isocupressic acid [b] [15-hydroxy-8(17), E-13, 19-labdadienoic acid], acetylisocupressic acid [c] [15-acetyloxy-8(17), E-13, 19-labdadienoic acid], imbricatoloic acid [d] [acid 15-hydroxy-8(17)-19-labdenoic acid] and comunic acid [e] [8(17),12,14-19 labdatrienoicoacid] (mixture of cis-trans isomersuma, cis:trans 1:2, in accordance to ¹H-NMR). These acids are components of oleoresins, particularly of Araucaria species, original of southern region of Brazil, where this sample was collected. These diterpenic acids were firstly insulated from propolis samples and the result are presented in the following publication (Bankova et al., Z.Natufforschung., 51B, 735-737, 1996). Antibacterial activity of the insulated compounds has been evaluated by means of the bioautography. Bankova et al., Z.Natufforschung, 50C, 167-172, 1995 found that said propolis sample, form which the insulated compounds have been extracted had antibacterial activity. This makes evident that the carboxylic groups at the C-19 and hydroxylic group at the C-15 position are important for the antibacterial activity of the insulated labdan acids: [b] e [d] were the most active in view of the derivatives [a] and [c], and also [e] which presents no OH group:

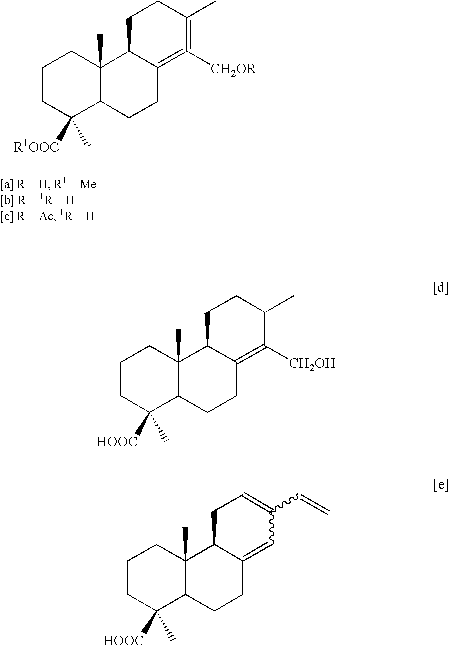

Structure of labdan type diterpenic acids

Other insulated compounds were [f] and [g]. After the obtention of mass spectra and ¹H-NMR, the structure of [f] has been clarified, and corresponds to the 10,11-di-o-acetyl deshydrodiconiferilic alcohol. This is a lignan, firstly reported as present in natural products. Furthermore, other compound insulated from the same sample is a flavonoid identified as being kaempferide [g] (4′-o-methyl-kaempferol), by compairson with the literature data (Bankova et al, Z. Naturforschung, 51B, 735-737, 1996).

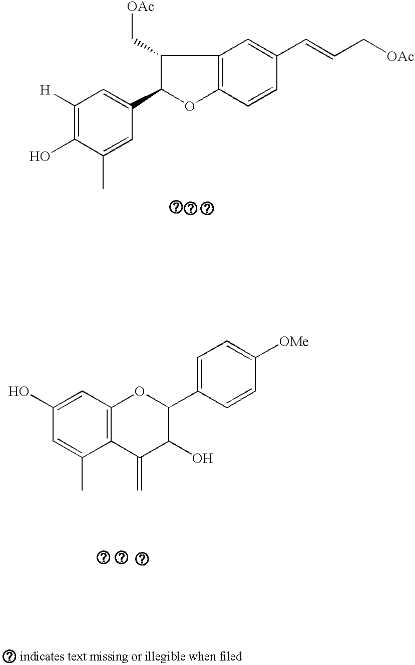

Strucuture of the compounds f and g id ntified from Brazilian propolis

Novel diterpenic acids with remarkable biological activity were identified as follows: a diterpenoid clerodan with antitumor activity (Matsuno, Z.Naturforschung., 50C, 93-97, 1995), the citotoxic substances: 15-oxo-3,13 Z-17-colavadienoic acid and its isomer E (Matsuno et al., Z.Naturforschung., 52C, 702-704, 1997).

These reported results shows remarkable differences between Brazilian propolis and the others (FIG. 3). Therefore, the development of one or more methods of Brazilian propolis is crucial. First, it has been though that the samples have several differences, varying from one region to other region in Brazil, after, due to the HPLC methodology innovation, it has been concluded that the Brazilian samples are divided through great Brazilian geographic regions, and forming three principal groups which are classfied only by means of this methodology. Accordingly, this method is able to determine a chemical profile of different samples and determine a quality standard. Also, the above method is able to identify the Brazilian propolis marker, the 3,5-diprenyl4-hydroxycinnamic acid.

Referring to the Brazilian própolis, some patents have been granted and the more relevant are as follows:

1. “Pure propolis extract”: The extract is prepared by several solvent extraction and the product is directed to cosmetic, pharmaceutical and oral hygiene industries (Hamada et al., U.S. Pat. No. 5,529,779, 1996).

2. “Propolis extract”: reduction of própolis aroma aiming the manufacture of food, cosmetic and pharmaceutical products as: antiseptics, preservatives, antifungicals e antioxidants (Shibuya et al., U.S. Pat. No. 6,153,227, 2000).

The European Patent Bank (some US are included), comprising 300 documents and the USPTO documents (137) have been examined; There are no similar patented process in the examined state of the art. The documents of the art generally report the production of pharmaceuticals, cosmetic and oral hygiene propolis based products and some derivative compounds having therapeutical and biological activities disclosed.

### BRIEF DESCRIPTION OF FIGURES

FIGS. 1 and 2: Chromatography data of similar compounds; the separation has effected λ=280 nm (as in experimental part)—1: caffeic acid, 2: p-coumaric acid, 3: ferulic acid, 4: pinebanksin, 5: PHCA, 6: DCBEN, 7: DHCA, 8: E (unknown), 9: DPB;

FIG. 3: Chromatography data of similar samples having the separation done at λ=280 nm , (as in experimental part); peaks indicated letters are related to the insulated compounds of the same sample, and quantified in other samples where they are present;

FIG. 4: Chromatography data of similar samples having the separation done at λ=280 nm (as in experimental part). 1: unknown, 2: kaempferol, 3: apigenin, 4: esther from 4-hydroxybenzoic acid, 5, 6 and 7: esthers from p-coumaric acid.

FIG. 5: Complex flavonoid-aluminum formation detected at 425 nm;

FIG. 6: UV spectra of the HPLC indentified compounds under the previous described conditions: PHCA is the 3-prenyl4-hydroxycinnamic acid, DHCA is the 3,5-diprenyl-4-hydroxycinnamic acid and E is an unknown compound, but its NMR spectrum does not indicate a flavonone compound. PHCA and DHCA have been insulated and identified by NMR (H and C) and MS;

FIG. 7: UV spectra of the HPLC identified compounds under the above described conditions: DCBEN is the 2,2-dimethyl4-carboxiethenyl-2H-1-benzopyran (a); the DCBEN compound has been insulated and identified by NMR (H and C) e MS; crisine (b), by means of procedure under the same conditions, has a very similar resulting retention time; DPB is the propenoic-2,2-dimethyl-8-prenyl-2H-1-benzopyran acid (c), insulated and identified by NMR (H and C) and MS; the tectocrisine (d), by means of procedure under the same conditions, has a very similar resulting retention time; on left: identification in Brazilian propolis, (a) and (c); on right: identification in European propolis and in other parts of World (b) and (d) (Caro, 1995);

FIG. 8: UV spectra of HPLC identified compounds, under the above described conditions. K is unknown, but the study of its structure does not indicate a flavanoid. The K compound is being identified by NMR (H and C) and MS. By means of the same physical measurement, the I compound has been identified as: 2-[1-hydroxymethyl]vinyl-6-acetyl-5-hydroxycumaran. Quercetin and luteolin flavonoids, under the same conditions, have very similar retention times of the above compounds;

FIG. 9: Chemical structure of the prenyl residue;

### DETAILED DESCRIPTION OF THE INVENTION

The process of the present invention comprises extraction of the raw material by honeycomb scraping. A micro scale amount of the material (ranging from 50 to 150 mg) has been dissolved in hot methanol (the volume ranging from 1.0 to 5,0 mL), filtered in filter paper and after passed in a Millipore filter for further analysis. When the sample was liquid, it has been employed as obtained by applying a dilution factor as follows: 1, 2, 3, . . . 10% diluted in relation to the original extract.

For complex sample separation the work is done in chromatography column. The analysis has been run in a liquid chromatography apparatus having a photodiode lattice and an automatic injector. The chromatography conditions are: movable phase water-formic acid (95:5, A solvent) and methanol chromatographic grade (B solvent). Eluition has run flowing at 1 mL/min by means of a linear gradient. The total analysis time was 50 minutes and detection occured at 280 e 340 nm wavelenghts.

Some reference compounds have been also run with the sample in a chromatography analysis. (García-Viguera et al., Z.Naturforschung., 47C, 634-637, 1992; Z.Naturforschung., 48C, 731-735, 1993). Other compounds, not commercially available but sythesized and characterized by author, has also served as reference. The present invention is associated to a specific software for data treatment of the results which is disclosed at the end of the present specification.

Other technique applied in the separation process is the Gas Chromatography coupled to the mass spectrometry, GC-MS. 5-10 mg of dry methanolic propolis extract were dissolved in 10-20 μL of pyridine and after 100 μL of reactive bis-(trimethylsilil)trifluoroacetamide (BSTFA) containing trimethylchlorinesilane (TMCS) 1%. The mixture was incubated at 100° C. during 30 minutes in a closed tube. Derived samples were separated and GC-MS analysed under the following conditions: HP 5 (phenylmethysilicone, Hewlett-Packard) capilar columnes 30 m, 0.25 mm-inner diameter (mass specific), carrier gas-He, solvent delay of 4 minutes, mass detector temperature: 280° C., injector temperature: 250° C., analysed mass ranging: between 40 and 650 u.m.a., flow: 1 ml/min, split relation: 1/100, pressure: 0.73 bar (100° C.), temperature biasing: 100° C. (initial), 2 min, velocity: 8° C./min up to 200° C. (2 min), 5° C./min up to 310° C. (maximum) during 6 min. Separation is done in 40 minutes.

Chromatographic methods of literature are very similar to the methods of the invention, but for the comprehension and quantification of the peaks of the chromatography results, as seen on apended Figures, standard reference compounds, insulated from the Brazilian samples and inherent to said samples are necessary, one of them is the principal chemical marker of the samples. Therefore, even similar conditions of HPLC chromatographic separation are employed the use of standard references (prepared by the author) for identification and quantification of the samples are necessary in order to obtain the same typing. Thus, this set of experimental conditions: column, movable phase, gradient and standard references for compairson and identification is the makes the present invention novel for not having similar identification, quantification and typing method in the literature of the art related to Brazilian propolis samples. The GC-MS analysis resulted in an excellent complement to the HPLC.

After the HPLC analysis and quantification, the samples have been statistically analysed by means of a multiple variation analysis software.

The principal component which is responsible for the sample separation is the PC1, PC1 contains the major part of the information. In the Figures, it is possible to notice that the samples having lower content of the most important bioactive compounds are placed in the quadrant where the PC1 is negative, samples having higher contents of said compounds are placed, obeying an increased ordinal from zero, in the quadrant where PC1 is positive. It is also noticed that the bioactives compounds are placed in positive PC1 quadrant.

Therefore, 3 from 100 samples analysed by the multiple variation software have the following features: one group having high content of bioactive compounds (BRP1), samples containing intermediary content (BRP2) and samples having lower contents (BRP3). There is no clear separation between the samples having lower and intermediary contents (separation near to zero), but it is clearly noticed a well defined group of high content samples.

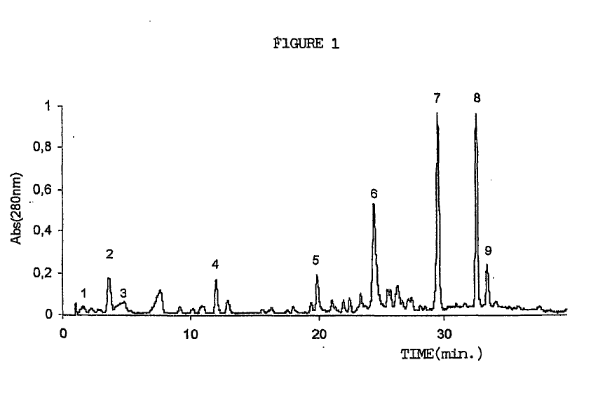

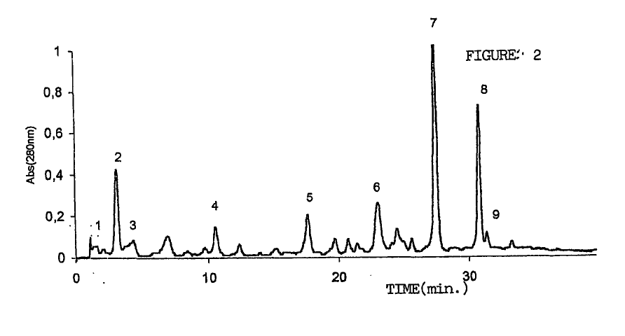

Group 1: FIGS. 1 and 2 show a chromatographic profile of the sampled of said group, the principal one, by containing the major part of the analysed propolis. Compounds analysed and quantified (in mg/g of raw sample or mg/mL of propolis extract) in the different samples of this group are: caffeic (Caf) acid, p-coumaric (p-C) acid, ferulic (Fer) acid, and pinobanksin acid (Pink) (flavonoid) (identified by means of compairson of known standard references), caffeic acid (Caf1, Caf2, Caf3, Caf4, Caf5) derivatives (identified for having the same UV spectrum but having differ nt retention times in relation to the caffeic acid being quantified by the caffeic acid chromatogram area), DHCA derivatives (DHCA1, DHCA2, DHCA3, DHCA4, DHCA5, DHCA6, DHCA7, DHCA8, DHCA9 e DHCA10) (the same UV spctrum of the 3,5-diprenyl4-hydroxycinnamic acid (DHCA), but having different retention times in relation to the DHCA acid, being quantified by DHCA acid chromatogram area), PHCA, DCBEN, DHCA, E (unknown) and DPB compounds (identified by compairson to insulated standard references).

Accordingly, the present invention made the relation between the vegetal origin and the geographic origin of the samples and the presence, or absence, of said substances. Some cases of group 1 and group 2 substances containing samples detection are also reported.

Table X values measured in mg/mL of the components quantified by HPLC chromatograms. Higher values of the DHCA component and also values of PHCA, DCBEN, E and DPB. For compairson ends, data of Table XI are converted in percent values by using the following formulae:

Value(%)=[mg/g(or mg/mL)]/10

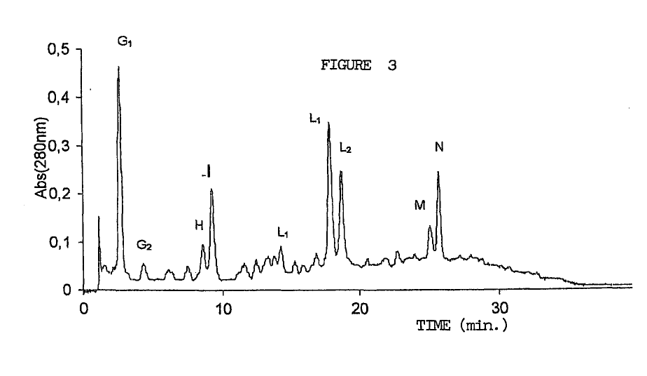

Group 2: FIG. 3 shows the chromatographic profile of the samples of the second group of similars. The retention time and the respective indentified compounds of said sample are presented in FIGS. IV and IX.

This sample group is totally different from the prior group, only some group one compounds have been found and in other samples the referred compounds are totally absent. In this case, all compounds were insulated and quantified, except the galic acid due to its very reduced peak. The concentration of the above mentioned compounds is different in the samples and in some cases the compounds are not present. As seen in the Table XVII, the conclusion to be reached is that all samples of the second group do not have origin in vegetal sources containing Baccharis dracunculifolia, the principal vegetal source of the first group. Some of said compounds has no completely clear chemical strucutures, but efforts, physical methods, are made in this sense. Only the compound I has crystalline form (yellow), the other compounds are brown resins. G2 and I compounds have their structures described a and are identified as pertaining to this group.

Table XVI shows values in mg/mL of the quantified of the HPLC chromatograms. Intermediary values of DHCA concentration are observed and also to the PHCA values (totally absent in table samples), DCBEN, E and DPB. The compairson to Table X can be directly done.

Values presented in XVI refer only to a little percent part of the presently analysed samples, therefore they are not totally presented.

Values of Table X refer only to a little percent part of the presently analysed samples, therefore they are not totally presented.

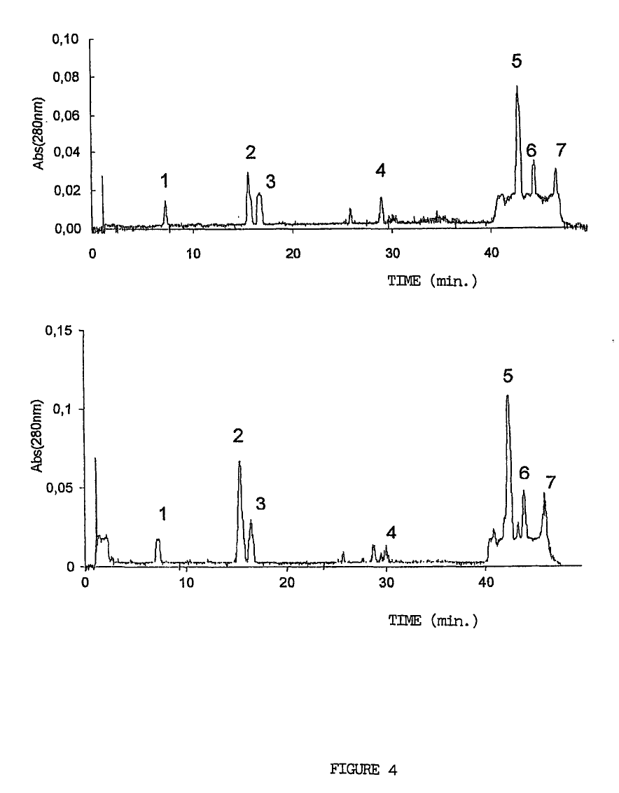

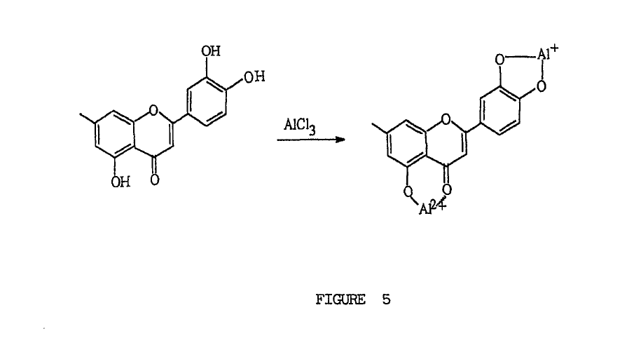

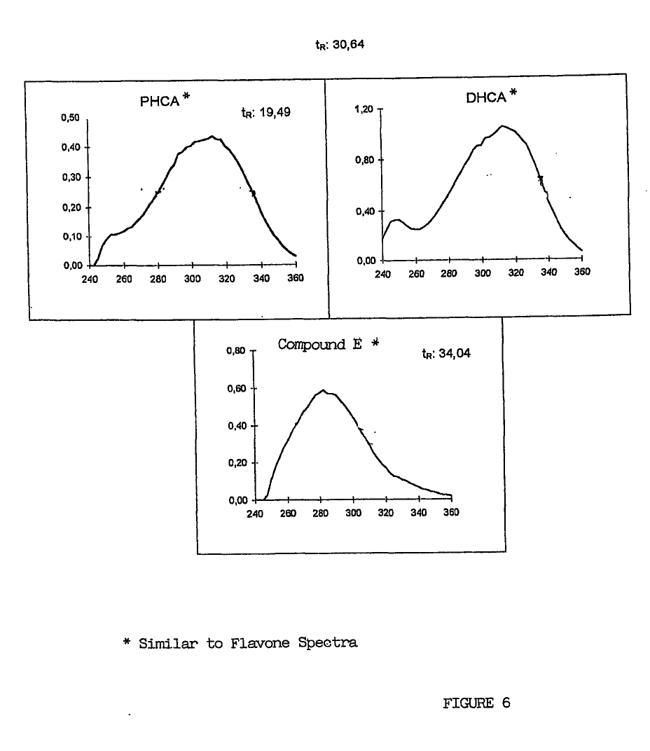

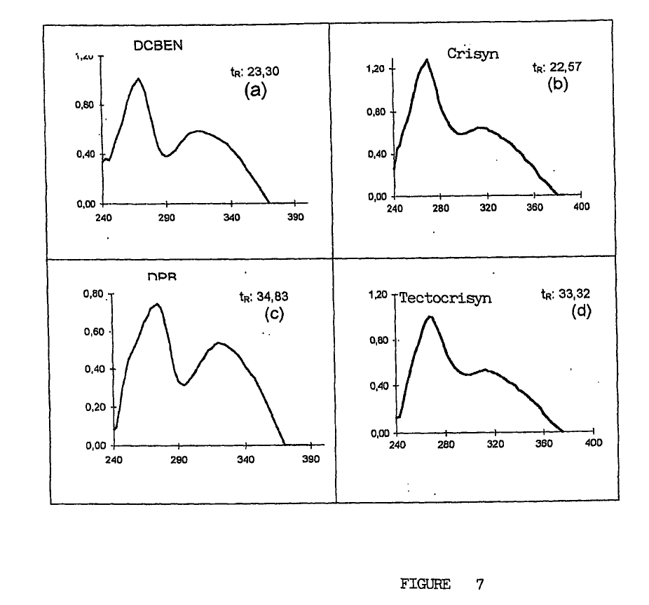

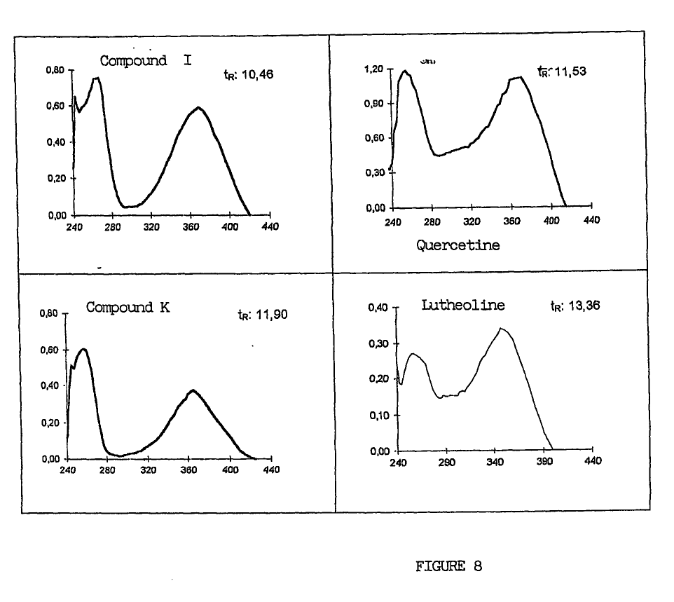

Group 3: This group contains a little number of samples and have its chromatographic profile described in Table I. HPLC rendered a few informationdue to the fact that said sample are hard to be dissolved in methanol, being very aromatic. Kaempferol and apigenin, a 4-hydroxybenzoic acid derivative and three p-coumaric acid derivative, compared to existing standard references. The amount of said flavonoids are very low in all propolis of this group, the samples present very low absorbance results (FIG. 4), when compared to the other two groups, at the same concentration (Tables XVII and XVIII). A sample of this group was further GC-MS analysed and it resulted 11 peaks.

As seen in Table XVI, the values of the phenolics compounds are lower than in the other groups.

This kind of liquid chromatography analysis is important due to the present method is able to well separate each UV absorbing component and by means of the GC-MS, it is possible the identification other non UV absorbing compounds, like the terpenes, largely present in Brazilian propolis and having remarked biological activity.

The HPLC method is able to identify major amounts of the Brazilian propolis components. The great limitation to improve a efficient and safe propolis quality control is the not correct identification of flavanoids presence in American and European samples (Table III), which are employed in Brazil as standard references but said compounds are pratically absent from the Brazilian propolis composition. Some insulated and identified compounds give positive reaction with aluminum chloride, the reagent for total flavonoids quantification (routine methodolgy), but they are not flavonoids (see compound I). Therefore, the quantification of total flavonoids by means of the methodology of the art (based on the German pharmacopeia) is not suitable to quantify the compounds of the Brazilian propolis, since, as referred above, these compounds have positive reaction without being flavonoids. FIG. 4 shows a flavonoid-aluminum complex (from Aluminum chloride) (Marcucci et al., Mensagem Doce, no. 46, 3-8, 1998).

UV spectra illustrate the previous related fact, the flavonoids quantification in Brazilian propolis, flavonoids are pratically absent from the Brazilian propolis samples. This result is only based on the chromatographic analysis.

GC-MS Dropolis analysis:

From the three groups, two samples of group one (No 21 and 23), one sample of group two (17) and one of the group three (44), were collected for this evaluation. From the first group two samples were chosen for having very similar HPLC chromatographic profiles and different antibacterial activities (FIA, flow injection analysis, E.coli ATCC 25922, 120 minutes of incubation, propolis concentration: 2 mg/mL). The sample 21 did not present toxity and the sample 23 presented 31% of culture growing inhibition. Since the samples were chosen, the gas chromatography separation condition was set as presently described. In the first sample, (17), 82 peaks were detected, the corresponding mass spectrum are not presently described. Although the great amount of peaks, only 10 compounds, some hydrocarbons, fat acids, vaniline and two amiryn isomers were identified by compairson to previous studied Brazilian propolis, (Table XVIII).

Sample 23, presented 90 peaks in its chromatographic separation, but only 28 were identified and presented some compounds different from the sample 21 as seen in Table XI. It can be noticed that even the latter has 22 peaks lesser than the sample 21, it presents antibacterial activity greater than the former, due to one substance or a little group of substances not pertaining to group 21, which is confirmed by GC-MS.

This technique revealed as being an excellent complement to the HPLC sample analysis. Chemical Marker:

For the groups characterized in the liquid chromatography, one could determine a profile based on the occurrence of PHCA, DCBEN, DHCA, E and DPB compounds, which can serve as propolis markers characterized by the presence of said compounds in pratically all analysed samples.

After injection of over 1700 samples in the chromatograph, the conclusion to be reached is that the Brazilian propolis has a marker, i.e., a majority component which appears in great part of the analysed samples, namely, , the 3,5-diprenyl4-hydroxycinnamic acid (DHCA) commercially called Artepillin C® by Japan and sold as a powder, said compound being employed in tumor treatment. This is the real marker of the Brazilian propolis.

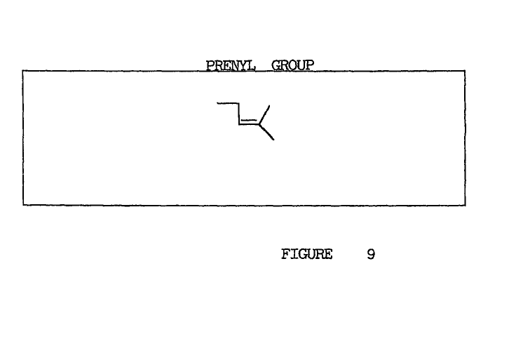

The marker compound was firstly insulated by Aga et al. (Biosci.Biotech.Biochem., 58, 945-946, 1994) from a Brazilian propolis sample. Said authors have compared the antibactarian activity of the 3,5-diprenyl-4-hydroxycinnamic acid to the 3-prenyl-4-hydroxycinnamic acid (PHCA) and 4-hydroxycinnamic acid (p-coumaric), concluding that the activity of said class of compounds can be enhanced as the number of prenyl residues increases. (the structure described in FIG. 9).

The same authors have tested the DHCA activity against skin-funges like Microsporeum gypseum (minimal inhibitory concentration, MIC: 7,8 μg/mL), A.benhamiae (MIC: 15,6 μg/mL) and bacters: B.subtilis (CIM: 31.3 μg/mL), Corynebacterium equii (CIM: 31.3 μg/m/L), Micrococcus lysodeikticus (CIM: 31.3 μg/mL), Pseudomonas aeruginosa (MIC: 31.3 μg/mL), Mycobacterium smegmatis (MIC: 31.3 μg/mL), Mycobaterium phlei (MIC: 62.5 μg/mL), Staphylococcus aureus (MIC: 62.5 μg/mL), Staphylococcus epidermidis (CIM: 62.5 μg/mL) e Thermoactinomyces intermedius (MIC: 62.5 μg/mL), indicating that this compound has effected a relatively strong activity against these microorganisms (Aga et al., Biosci.Biotech.Biochem., 58, 945-946, 1994). Banskota et al., (J.Nat.Prod., 61, 896-900, 1998) reported DHCA activity in tumor cells, with ED₅₀values (cytotoxic dosage—50%): 45.47 μg/mL against fibrosarcoma (line HT-1080) and 59,32 μg/mL against uterus colon carcinoma (line L5-26), the methanolic extract of propolis presented ED₅₀=67.33 μg/mL (HT-1080) e 62.35 μg/mL (L5-26).

### Marker based Typing

Based on the obtained data and related to the marker, the Brazilian propolis typing procedure is as follows: BRP type, with three subdivisions:

BRP 1: High marker concentration: DHCA (from 15 to 40 mg/g or 1.5 to 4.0%) E (from 40 to 90 mg/g or 4.0 to 9.0%) DPB (from 15 to 40 mg/g or 1.5 to 4.0%) P-CUM (from 7 to 15 mg/g or 0.7 to 1.5%) PHCA (from 8 to 20 mg/g or 0.8 to 2.0%) PINK (from 8 to 25 mg/g or 0.8 to 2.5%).

BRP 2: Intermediary marker concentration: DHCA (from 10 to 15 mg/g or 1.0 to 1.5%) E (from 20 to 60 mg/g or 2.0 to 6,0%) DPB (from 4 to 15 mg/g or 0.4 to 1.7%) P-CUM (from 3 to 9 mg/g or 0.4 to 1.0%) PHCA (from 2 to 8 mg/g or 0.2 to 0.8%) PINK (from 2 to 8 mg/g or 0.2 to 0.8%).

BRP 3: Low marker concentration: DHCA (from 1 to 10 mg/g or 0.1 to 1.0%) E (from 2 to 20 mg/g or 0.2 to 2.0%) DPB (from 1.0 to 10 mg/g or 0.1 to 1.0%) P-CUM (from 0.25 to 5 mg/g or 0.025 to 0.5%) PHCA (from 0.25 to 4 mg/g or 0.025 to 0.4%) PINK (from 0.1 to 5 mg/g or 0.01 to 0.5%).

One group of similar samples, but different from the first group, containing compounds G₁, G₂, H, I, J, K, L₁, L₂, M and N, is comprised by propolis from Southern part of Brazil therfore typed as: BRG type, having three subdivisions:

BRG 1: Intermediary submarker* (G₁) concentration and low concentration of the DHCA marker and the same in relation to the compounds: PHCA, DCBEN and DPB.

BRG 2: Intermediary-to-Low concentration of the submarker* (G₁) and low concentration or absence of DHCA marker and the same in relation to the compounds: PHCA, DCBEN and DPB.

BRG 3: Low concentration of the submarker* (G₁) and low concentration or absence of the DHCA marker and the same in relation to the compounds: PHCA, DCBEN and DPB.

The principal feature of this typing is the fast conduction of the propolis from the farm to the industry for permitting to the latter employ the previous typed product directly in pharmaceuticals manufacture.

Another important feature of the typing is produce cosmetics, pharmaceuticals, oral hygiene and “health foods”, having previous detailed the propolis composition: type of propolis, amounts of bioactive components, and propolis features never disclosed before in patent bank.

Data Treatment Software:

The HPLC-Propolis system is a software which works inside a Windows Operational System, proposed for work as a data treatment tool of the information collected from chromatograph output.

Data input of this software is carried out by means of a file generated in the chromatograph by user typed information and programmed information originally present in the software.

The chromatograph file is read by the software and their information is inserted into a data bank. The read information refers to the compounds present in the propolis sample: compound code, column retention time, and chromatograph area of the compound.

The typed information are: user name, user password, name or number of the chromatogram, name or number of the sample, origin, botanic origin, collection date, aspect of the sample, injected volume in the chromatograph, dilution, etc.

Recorded information are; compound code, complete compound name, column retention time and chromatograph area of the compound, these data refer to the standard reference.

The information processing comprise calculation of concentration of each compound present at the separation (chromatogram) of a propolis sample. This calculation considers the peak area of the chromatogram in relation to the standard area which the concentration is known. A dilution factor is included in the calculation. The result (of the chromatogram peak concentration) is inserted again in the software data bank for consulting. These data referring to the amount of each compound are displayed in an output Table and finally the sample typing is concluded, dependending on the concentration of its principal components.

The information output is a report generated by the software. The report contains the following information: chromatogram name or number, sample name or number, the compound code, compound complete name, column retention time of the compound, the concentration obtained by the previous proceeding and the typing group of the sample. This report can be seen at the computer screen or ever paper printed. This data analysis system permits new standard reference insertion, when said references were identified in propolis samples. The system also permits the same quantification and typing determination of any kind of natural product if it is analysed by HPLC or by GC-MS, since the chemical structures of the active components are known.

TABLE I
Antibacterial activty of the compounds (DCBEN, DHCA,
E + DPB) (group 1) e G₁, G₂, K e L₂ (group 2).

| | COMPOUNDS | | | | | | | |
|---|---|---|---|---|---|---|---|---|
| Microorganisms | DCBEN | DHCA | E | E + DPB | K | G₁ | G₂ | L₂ |
| Escherichia coli | + | Ø | + | ++ | + | + | + | + |
| Pseudomonas | Ø | Ø | Ø | + | Ø | Ø | Ø | + |
| aeruginosa | | | | | | | | |
| Staphylococcus | ++ | + | + | +++ | + | + | ++ | + |
| aureus | | | | | | | | |
| Streptococcus | ++ | + | + | ++ | + | + | ++ | + |
| faecalis | | | | | | | | |

+ halo < or = to 8 mm
++ halo > or = to 8 mm and < 12 mm
+++ halo > to 12 mm

TABLE II
Effects of the insulated propolis compounds against
T. cruzi trypomastigotes (in mg/mL).

| COMPOUND | ID₅₀/24 h (mg/ml) |
|---|---|
| DCBEN | 1.696 ± 0.032 |
| DHCA | 0.932 ± 0.046 |
| E | 2.036 ± 0.092 |
| E/DPB | 0.942 ± 0.046 |
| Cristal violet | 0.205 |
| EEP* | 2.5 |

EEP: Ethanolic Propolis Extract from which the Table compounds were Insulated.

TABLE III
Propolis compounds from different regions*.

| Geographic origin | Fonte vegetal | Principal components |
|---|---|---|
| Europe, Asia, North | Populus spp. | Flavonoids: pinocembrin, |
| America | | pinobanksin, pinobanksin-3-O- |
| | | acetate, crisyn, galangine, |
| | | aromatic acids: caffeic, ferulico |
| | | and caffeates: benzyl, |
| | | phenylethyl e prenyl). |
| Russia (nothern) | Betula verrucosa | Flavonoids: acacetine, |
| | | apigenin, ermanin, |
| | | ramnocitrine, kaempferide, |
| | | others: α-acetoxybetulenol. |
| Brazil | Baccharis spp. | prenyl p-coumaric acids, prenyl |
| | Araucaria spp. (?) | acetophenones and diterpenic |
| | | acids, others. |
| Canary Islands | Unknown | Furofuranic Lignans |

*From Bankova et al., 2000.

TABLE IV
Abbreviations and names corresponding to each
HPLC identified compound in accordance to their retention
time (minutes) of group1.

| Abbrev. | Compound (quantified in mg/mL* or mg/g**) |
|---|---|
| CAF | Caffeic acid (d) |
| P-CUM | p-coumaric acid (d) |
| FER | Ferulic acid (d) |
| CAF1 | Caffeic acid(derivative 1) |
| CAF2 | Caffeic acid (derivative 2) |
| DHCA1 | 3,5-diprenyl-4-hydroxycinnamic acid (derivative 1) |
| PINK | Pinobanksin (i*) |
| CAF3 | Caffeic acid (derivative 3) |
| DHCA2 | 3,5-diprenyl-4-hydroxycinnamic acid (derivative 2) |
| DHCA3 | 3,5-diprenyl-4-hydroxycinnamic acid (derivative 3) |
| PHCA | 3-prenyl-4-hydroxycinnamic acid (i) |
| DHCA4 | 3,5-diprenyl-4-hydroxycinnamic acid (derivative 4) |
| DCBEN | 2,2-Dimethyl-6-carboxiethenyl-2H-1-benzopyran (i) |
| DHCA5 | 3,5-diprenyl-4-hydroxycinnamic acid (derivative 5) |
| KAEMP1 | Kaempferol (derivative 1) |
| DHCA6 | 3,5-diprenyl-4-hydroxycinnamic acid(derivative 6) |
| DHCA7 | 3,5-diprenyl-4-hydroxycinnamic acid (derivative 7) |
| DHCA8 | 3,5-diprenyl-4-hydroxycinnamic acid (derivative 8) |
| DHCA9 | 3,5-diprenyl-4-hydroxycinnamic acid (derivative 9) |
| DHCA10 | 3,5-diprenyl-4-hydroxycinnamic acid (derivative 10) |
| DHCA | 3,5-diprenyl-4-hydroxycinnamic acid (i) |
| E | Compound E (i**) |
| PCB | 6-propenoic-2,2-dimethyl-8-prenyl-2H-1-benzopyran acid |
| | (i) |
| DHCA11 | 3,5-diprenyl-4-hydroxycinnamic acid (derivative 11) |
| CAF4 | Caffeic acid(derivative 4) |
| CAF5 | Caffeic acid(derivative 5) |

*liquid sample;
**solid sample.
(d): commercially available;
(i*): insulated by Dr. Francisco Tomas-Barberan group, Spain;
(i): Insulated by the author;
(i**): insulated by the author but not identified at the present.

TABLE IX
Abbreviations and names corresponding to each
HPLC identified compound in accordance to their retention
time (minutes) of group 2.

| Abbrev. | Compound (quantified in mg/mL* or mg/g**) |
|---|---|
| GA | Galic acid (d) |
| HBENZ | 4-hydroxybenzoic acid (d) |
| DHBENZ | 2,4-dihydroxybenzoic acid (d) |
| G1 | Compound G1(i*) |
| P-CUM | p-coumaric acid (d) |
| FER | Ferulic acid (d) |
| G2 | 3-methoxy-4-hydroxycinnamaldehyde (i) |
| I | 2-[1-hydroxymethyl]vinyl-6-acetyl-5-hydroxy-coumaran (i) |
| H | Compound H (i**) |
| K | Compound K (i**) |
| PHCA | 3-prenyl-4-hydroxycinnamic acid (i) |
| L2 | Compound L2 (i**) |
| L1 | Compound L1 (i**) |
| DCBEN | 2,2-Dimethyl-6-carboxyethenyl-2H-1-benzopyran (i) |
| KAEMP1 | Kaempferol (derivative 1) |
| M | Compound M (i**) |
| N | Compound N (i**) |
| DHCA | 3,5-diprenyl-4-hydroxycinnamic acid (i) |
| E | Compound E (i**) |
| PCB | 6-propenoic-2,2-dimethyl-8-prenyl-2H-1-benzopyran |
| | acid(i) |

*liquid sample;
**solid sample.
(d): commercially available;
(i*): insulated by Dr. Francisco Tomas-Barberan group, Spain;
(i): insulated by the author;
(i**): insulated by the author but not identified at the present.

TABLE XI
Apigenine and Kaempferol compositions in propolis
samples presenting similar chromatographic profile.
Values expressed in mg/g of sample a percent (%)
(by weight per gram of sample).

| | Apigenine | Apigenine | Kaempferol | Kaempferol |
|---|---|---|---|---|
| | (d) | (d) | (d) | (d) |
| Sample | (mg/g) | (%) | (mg/g) | (%) |
| 1 | 0.50 | 0.05 | 1.34 | 0.13 |
| 2 | 0.49 | 0.05 | 1.77 | 0.18 |
| 3 | 2.01 | 0.20 | 4.57 | 0.46 |
| 4 | 0.79 | 0.08 | 4.04 | 0.40 |
| 5 | 0.00 | 0.00 | 0.00 | 0.00 |
| 6 | 0.45 | 0.05 | 1.95 | 0.20 |
| 7 | 0.66 | 0.07 | 1.85 | 0.19 |
| 8 | 1.63 | 0.16 | 2.76 | 0.28 |
| 9 | 1.18 | 0.12 | 3.47 | 0.35 |
| 10 | 0.96 | 0.10 | 2.64 | 0.26 |
| 11 | 2.04 | 0.20 | 7.71 | 0.77 |

TABLE XII
Relation of the GC-MS identified compounds in
the sample No. 17, and their respective retentio times.

| | Retention Time | |
|---|---|---|
| N°. | (tᵣ) in minutes | Identified compounds |
| 1 | 8.534 | Vaniline |
| 2 | 18110 | Hexadecanoic acid (palmitic) |
| 3 | 19.735 | 9-octadecenoic acid (oleic) |
| 4 | 25.629 | Pentaeicosane |
| 5 | 26.580 | Octahydro-1,4,9,9-tetramethyl-1H-3A,7- |
| | | methane azulene |
| 6 | 30.151 | Docosane |
| 7 | 33.745 | 1-Docosene |
| 8 | 36.122 | (2)-9-Tricosene |
| 9 | 37.316 | α-Amiryn |
| 10 | 37.637 | β-Amiryn |

TABLE XIII
Relation of the GC-MS identified compounds of
sample No. 21, and their respective retention times.

| | Retention | |
|---|---|---|
| | Time (tᵣ) in | |
| n°. | minutes | Identified compounds |
| 1 | 6.711 | Glycerol ether |
| 2 | 7.277 | Butadienoic acid |
| 3 | 8.195 | Styrene |
| 4 | 8.837 | Benzenepropanoic acid |
| 5 | 9.622 | 4′-hydroxyacetophenone |
| 6 | 10.071 | Butanodioic acid |
| 7 | 10.153 | Malic acid |
| 8 | 10.363 | 4-methoxybenzoic acid |
| 9 | 10.525 | 3-methoxy, 4-hydroxybenzaldehyde |
| 10 | 10.606 | Vaniline |
| 11 | 11.447 | 2,3-dihydroxybutanoic acid |
| 12 | 11.966 | 4-hydroxybenzoic acid |
| 13 | 13.791 | p-hydroxydihydrocinnamic acid |
| 14 | 13.857 | 3-methoxy-4-hydroxybenzoic acid |
| 15 | 14.167 | Cis-p-coumaric acid |
| 16 | 14.587 | 3-Vanilinpropanol |
| 17 | 14.842 | 3-Hydroxy-4-methoxycinnamaldehyde |
| 18 | 14.919 | D-Frutose |
| 19 | 15.649 | 3-methoxy-4-hydroxybenzenopropanoic acid |
| 20 | 16.069 | Hexose |
| 21 | 16.174 | p-hydroxycinnamic acid |
| 22 | 16.357 | trans p-coumaric acid |
| 23 | 17.031 | Propyl p-Coumarate |
| 24 | 17.142 | Neo-inositol |
| 25 | 17.673 | Hexose |
| 26 | 18.883 | 3-methoxy-4-hydroxycinnamic acid |
| 27 | 19.011 | Ferulic acid |
| 28 | 19.586 | Mio-inositol |
| 29 | 19.907 | Caffeic acid |
| 30 | 21.001 | [Z,Z] 9,12-Octadecadienoic acid |
| 31 | 21.112 | 11-cis-octadecenoic acid |
| 32 | 21.532 | Octadecanoic acid (estearic) |
| 33 | 22.561 | Isopimaric acid |
| 34 | 22.807 | Butanedienoic acid |
| 35 | 22.863 | Hexadecanoic acid (palmitic) |
| 36 | 22.970 | Dihydroabietic acid |
| 37 | 24.300 | Abietic acid |
| 38 | 25.991 | Hexanedioic acid |
| 39 | 26.089 | Hydroxymethoxyflavone (tectocrisyn) |
| 40 | 26.907 | 1-Monoleilglycerol |
| 41 | 27.418 | Xylitol |
| 42 | 27.749 | Glycerol ether |
| 43 | 28.212 | 3,5-diprenyl-4-hydroxycinnamic acid |
| 44 | 28.654 | Glycerol |
| 45 | 29.253 | tetracosanoic acid (lignoceric) |
| 46 | 29.273 | Octadecanodioic acid |
| 47 | 30.137 | 3′,5,6-Triethoxy-3,4′,7- |
| | | trimethoxyflavone |
| 48 | 30.755 | Trihydroxy- |
| | | methoxyflavanone(pinobanksin) |
| 49 | 30.834 | Octadecanoic acid (estearic) |
| 50 | 31.751 | Tetrahydroxyflavone (kaempferol) |
| 51 | 32.934 | Dihydroxydimethoxyanthraquinone |
| 52 | 32.432 | Dihydroxymethoxyhydroxymethylanthraquinone |
| 53 | 34.471 | 3-prenyl-4- |
| | | dihydroxycinnamoylcinnamic acid |
| 54 | 35.090 | Caffeic acid esther |
| 55 | 35.621 | Triterpenic alcohol (amiryn type) |
| 56 | 35.677 | Triterpenic alcohol (amiryn type) |
| 57 | 36.064 | Triterpenic alcohol (amiryn type) |
| 58 | 36.506 | Triterpenic alcohol |
| 59 | 36.860 | Triterpenic alcohol (amiryn type) |
| 60 | 37.357 | Triterpenic alcohol (amiryn type) |
| 61 | 37.424 | Triterpene |
| 62 | 37.722 | α-Amiryn |
| 63 | 37.965 | m-Cinnamic acid methyl esther |

TABLE XIV
Relation of GC-MS identified compounds of sample No.
23, and their respective retention times.

| | Retention time (tᵣ) in | |
|---|---|---|
| n°. | minutes | Identified compounds |
| 1 | 6.670 | Glycerol ether |
| 2 | 8.756 | Benzenepropanoic acid |
| 3 | 10.047 | Butanodioic acid |
| 4 | 10.301 | 4-methoxybenzoic acid |
| 5 | 10.522 | 3-Methoxy-4-hydroxybenzaldehyde |
| 6 | 11.902 | 4-hydroxybenzoic acid |
| 7 | 13.723 | p-hydroxycinnamic acid |
| 8 | 14.098 | m-hydroxycinnamic acid |
| 9 | 14.584 | 3,4-dihydroxybenzoic acid |
| 10 | 14.650 | Sugar |
| 11 | 14.749 | Sugar |
| 12 | 14.826 | Xylose |
| 13 | 15.930 | D-galactose |
| 14 | 16.305 | P-coumaric acid |
| 15 | 17.034 | Neo-inositol |
| 16 | 17.177 | Hexose |
| 17 | 17.520 | Pentose |
| 18 | 17.950 | 2,4-dihydroxybenzoic acid |
| 19 | 18.016 | Hexadecanoic acid (palmitic) |
| 20 | 18.910 | Ferulic acid |
| 21 | 19.440 | Mio-inositol |
| 22 | 21.007 | Octadecanoic acid |
| 23 | 23.579 | Derivative of Ferulic acid |
| 24 | 24.363 | Abietic acid |
| 25 | 28.082 | β-D-Frutofuranose |
| 26 | 28.579 | Glycerol |
| 27 | 31.228 | Octadecanoic acid (estearic) |
| 28 | 31.681 | 3,4-dimethoxyhydroxycinnamic acid |

TABLE XV
Relation of GC-MS identified compounds of
sample No. 44, and their respective retention times.

| | Retention | |
|---|---|---|
| | time (tᵣ) in | |
| n°. | minutes | Identified compounds |
| 1 | 6.246 | Benzoic acid |
| 2 | 7.197 | 4-hydroxy-3-methoxyacetophenone |
| 3 | 7.595 | Benzenepropanoic acid |
| 4 | 8.258 | Coapene |
| 5 | 8.512 | Vaniline |
| 6 | 9.010 | Isobutyl cinnamate |
| 7 | 9.298 | Butyl cinnamate |
| 8 | 10.127 | Decahydro-1,1,7-trimethyl-4-methylene-1H- |
| | | cicloprop[E]azulene |
| 9 | 11.321 | Espatulenol |
| 10 | 11.542 | 2,6-Dimethoxy-4-(2-propenyl)phenol |
| 11 | 11.918 | Prenylacetophenone |
| 12 | 12.382 | 4-Hydroxy-3,5-dimethoxybenzaldehyde |
| 13 | 13.798 | Benzyl benzoate |
| 14 | 15.169 | Benzyl-2-phenylethylenylketone |
| 15 | 15.921 | Methyl Hexadecanoate |
| 16 | 16.750 | Hexadecanoic acid (palmitic) |
| 17 | 17.601 | Diprenylacetophenone |
| 18 | 18.895 | Benzyl Cinnamate |
| 19 | 19.603 | Methyl 4,7,10-Hexadecatrienoate |
| 20 | 19.946 | [Z] 9,17-Octadecadienal |
| 21 | 20.244 | Acido octadecanoic acid (estearic) |
| 22 | 22.422 | Tricosane |
| 23 | 23.760 | Heicosanoic acid |
| 24 | 24.048 | Hydroxyphenyl Benzoate |
| 25 | 25.198 | Heneicosanoic acid |
| 26 | 25.795 | Benzoic acid esther |

TABLE X
DHCA from 15 to 40 mg/g ou 1.5 to 4.0%. E from
40 to 90 mg/g ou 4.0 to 9.0%. PCB from 15 to 40 mg/g
or 1.5 to 4.0%. P-CUM from 7 to 15 mg/g or 0.7 to 1.5%.
PHCA from 8 to 20 mg/g or 0.8 to 2.0%. PINK from
8 to 25 mg/g or 0.8 to 2.5%.

| | Samples | | | | | | | | | | |
|---|---|---|---|---|---|---|---|---|---|---|---|
| Abbrev. | 32 | 36 | 87 | 88 | 90 | 92 | 93 | 94 | 98 | 99 | 100 |
| CAF | 0.96 | 1.08 | 0.65 | 0.00 | 0.55 | 0.04 | 0.67 | 0.92 | 0.43 | 0.76 | 0.58 |
| CAF1 | 0.00 | 2.53 | 8.55 | 0.00 | 2.19 | 5.30 | 0.57 | 2.62 | 2.29 | 1.87 | 3.74 |
| CAF2 | 0.00 | 0.00 | 1.07 | 0.00 | 0.00 | 0.56 | 0.00 | 0.19 | 0.00 | 0.00 | 0.49 |
| CAF3 | 0.00 | 0.00 | 0.00 | 0.00 | 0.00 | 0.00 | 0.00 | 0.60 | 0.00 | 0.00 | 0.00 |
| CIN1 | 0.00 | 0.00 | 0.00 | 0.00 | 0.00 | 0.00 | 0.00 | 0.00 | 0.00 | 2.08 | 0.09 |
| DCBEN | 0.00 | 17.56 | 1.68 | 0.00 | 1.37 | 0.00 | 0.00 | 1.56 | 0.68 | 0.79 | 2.36 |
| DHCA | 12.30 | 16.95 | 39.24 | 37.99 | 22.58 | 29.46 | 23.56 | 29.89 | 19.50 | 23.15 | 27.17 |
| DHCA1 | 0.00 | 1.30 | 2.31 | 0.76 | 0.12 | 0.56 | 0.00 | 0.70 | 7.78 | 0.69 | 0.76 |
| DHCA2 | 0.00 | 1.35 | 6.46 | 0.85 | 0.25 | 1.04 | 0.00 | 0.78 | 1.30 | 0.71 | 0.68 |
| DHCA3 | 0.00 | 5.31 | 0.36 | 0.50 | 1.08 | 0.46 | 0.00 | 0.15 | 0.17 | 0.93 | 0.23 |
| DHCA4 | 0.00 | 0.00 | 0.00 | 1.45 | 0.65 | 0.00 | 0.00 | 0.56 | 0.69 | 0.60 | 0.51 |
| DHCA5 | 0.00 | 0.00 | 0.00 | 0.46 | 0.50 | 0.00 | 0.00 | 0.00 | 0.31 | 2.52 | 2.04 |
| DHCA6 | 0.00 | 0.00 | 0.00 | 0.47 | 2.29 | 0.00 | 0.00 | 0.00 | 0.50 | 2.05 | 0.23 |
| DHCA7 | 0.00 | 0.00 | 0.00 | 0.00 | 0.66 | 0.00 | 0.00 | 0.00 | 2.15 | 0.89 | 4.12 |
| DHCA8 | 0.00 | 0.00 | 0.00 | 0.00 | 4.12 | 0.00 | 0.00 | 0.00 | 0.00 | 0.00 | 0.00 |
| DPB | 6.17 | 16.09 | 8.68 | 21.64 | 17.49 | 0.00 | 5.35 | 8.88 | 29.69 | 12.45 | 15.36 |
| E | 36.30 | 54.87 | 88.84 | 26.35 | 18.79 | 71.29 | 40.45 | 47.81 | 54.69 | 36.31 | 59.55 |
| FER | 0.00 | 2.87 | 2.81 | 0.00 | 0.57 | 3.12 | 0.00 | 0.00 | 0.40 | 0.33 | 0.70 |
| KAEMP1 | 17.66 | 11.40 | 20.88 | 17.28 | 13.42 | 49.58 | 4.37 | 16.44 | 16.77 | 11.54 | 13.19 |
| P-CUM | 3.56 | 6.78 | 13.09 | 9.97 | 7.57 | 4.37 | 7.28 | 13.18 | 7.18 | 4.58 | 9.57 |
| PHCA | 4.47 | 7.09 | 6.61 | 18.52 | 14.40 | 0.00 | 5.55 | 8.39 | 11.40 | 10.01 | 9.63 |
| PINK | 8.31 | 5.88 | 17.54 | 0.00 | 7.10 | 7.04 | 6.95 | 12.48 | 22.12 | 9.42 | 11.19 |
| CAF | 0.96 | 1.08 | 0.65 | 0.00 | 0.55 | 0.04 | 0.67 | 0.92 | 0.43 | 0.76 | 0.58 |
| CAF1 | 0.00 | 2.53 | 8.55 | 0.00 | 2.19 | 5.30 | 0.57 | 2.62 | 2.29 | 1.87 | 3.74 |
| CAF2 | 0.00 | 0.00 | 1.07 | 0.00 | 0.00 | 0.56 | 0.00 | 0.19 | 0.00 | 0.00 | 0.49 |
| CAF3 | 0.00 | 0.00 | 0.00 | 0.00 | 0.00 | 0.00 | 0.00 | 0.60 | 0.00 | 0.00 | 0.00 |
| CIN1 | 0.00 | 0.00 | 0.00 | 0.00 | 0.00 | 0.00 | 0.00 | 0.00 | 0.00 | 2.08 | 0.09 |
| DCBEN | 0.00 | 17.56 | 1.68 | 0.00 | 1.37 | 0.00 | 0.00 | 1.56 | 0.68 | 0.79 | 2.36 |
| DHCA | 12.30 | 16.95 | 39.24 | 37.99 | 22.58 | 29.46 | 23.56 | 29.89 | 19.50 | 23.15 | 27.17 |
| DHCA1 | 0.00 | 1.30 | 2.31 | 0.76 | 0.12 | 0.56 | 0.00 | 0.70 | 7.78 | 0.69 | 0.76 |
| DHCA2 | 0.00 | 1.35 | 6.46 | 0.85 | 0.25 | 1.04 | 0.00 | 0.78 | 1.30 | 0.71 | 0.68 |
| DHCA3 | 0.00 | 5.31 | 0.36 | 0.50 | 1.08 | 0.46 | 0.00 | 0.15 | 0.17 | 0.93 | 0.23 |
| DHCA4 | 0.00 | 0.00 | 0.00 | 1.45 | 0.65 | 0.00 | 0.00 | 0.56 | 0.69 | 0.60 | 0.51 |
| DHCA5 | 0.00 | 0.00 | 0.00 | 0.46 | 0.50 | 0.00 | 0.00 | 0.00 | 0.31 | 2.52 | 2.04 |
| DHCA6 | 0.00 | 0.00 | 0.00 | 0.47 | 2.29 | 0.00 | 0.00 | 0.00 | 0.50 | 2.05 | 0.23 |
| DHCA7 | 0.00 | 0.00 | 0.00 | 0.00 | 0.66 | 0.00 | 0.00 | 0.00 | 2.15 | 0.89 | 4.12 |
| DHCA8 | 0.00 | 0.00 | 0.00 | 0.00 | 4.12 | 0.00 | 0.00 | 0.00 | 0.00 | 0.00 | 0.00 |
| DPB | 6.17 | 16.09 | 8.68 | 21.64 | 17.49 | 0.00 | 5.35 | 8.88 | 29.69 | 12.45 | 15.36 |
| E | 36.30 | 54.87 | 88.84 | 26.35 | 18.79 | 71.29 | 40.45 | 47.81 | 54.69 | 36.31 | 59.55 |
| FER | 0.00 | 2.87 | 2.81 | 0.00 | 0.57 | 3.12 | 0.00 | 0.00 | 0.40 | 0.33 | 0.70 |
| KAEMP1 | 17.66 | 11.40 | 20.88 | 17.28 | 13.42 | 49.58 | 4.37 | 16.44 | 16.77 | 11.54 | 13.19 |
| P-CUM | 3.56 | 6.78 | 13.09 | 9.97 | 7.57 | 4.37 | 7.28 | 13.18 | 7.18 | 4.58 | 9.57 |
| PHCA | 4.47 | 7.09 | 6.61 | 18.52 | 14.40 | 0.00 | 5.55 | 8.39 | 11.40 | 10.01 | 9.63 |
| PINK | 8.31 | 5.88 | 17.54 | 0.00 | 7.10 | 7.04 | 6.95 | 12.48 | 22.12 | 9.42 | 11.19 |

DHCA from 10 to 15 mg/g or 1.0 to 1.5%. E form
20 to 60 mg/g or 2.0 to 6.0%. PCB from 4 to 15 mg/g
or 0.4 to 1.7%. P-CUM from 3 to 9 mg/g or 0.4 to 1.0%.
PHCA from 2 to 8 mg/g or 0.2 to 0.8%. PINK from
to 8 mg/g or 0.2 to 0.8%.

| | Samples | | | | | | | | |
|---|---|---|---|---|---|---|---|---|---|
| Abbrev. | 13 | 26 | 27 | 28 | 30 | 31 | 33 | 40 | 85 |
| CAF | 1.05 | 0.80 | 0.90 | 0.91 | 0.75 | 0.00 | 0.75 | 1.47 | 0.43 |
| CAF1 | 4.46 | 3.85 | 3.72 | 2.29 | 0.00 | 0.00 | 0.00 | 2.68 | 1.29 |
| CAF2 | 1.69 | 2.01 | 0.00 | 0.00 | 0.00 | 0.00 | 0.00 | 0.00 | 0.16 |
| DCBEN | 3.69 | 15.49 | 13.74 | 11.31 | 12.72 | 15.27 | 14.65 | 22.06 | 0.95 |
| DHCA | 12.46 | 14.71 | 16.06 | 11.36 | 13.40 | 10.67 | 14.77 | 11.74 | 16.43 |
| DHCA1 | 1.60 | 2.51 | 2.62 | 1.72 | 1.63 | 1.29 | 0.00 | 5.02 | 0.72 |
| DHCA2 | 1.61 | 4.79 | 0.00 | 0.00 | 3.75 | 0.00 | 0.00 | 5.29 | 0.33 |
| DHCA3 | 0.00 | 3.33 | 0.00 | 0.00 | 2.37 | 0.00 | 0.00 | 2.62 | 0.87 |
| DPB | 4.19 | 7.60 | 7.13 | 6.98 | 12.50 | 16.91 | 4.87 | 15.99 | 16.07 |
| E | 23.71 | 38.13 | 47.99 | 44.09 | 36.17 | 30.69 | 63.04 | 56.92 | 36.3 |
| FER | 2.81 | 2.53 | 2.29 | 0.00 | 0.00 | 0.00 | 0.00 | 3.12 | 0.34 |
| KAEMP1 | 18.11 | 14.70 | 18.66 | 0.00 | 0.00 | 0.00 | 0.00 | 0.00 | 12.26 |
| P-CUM | 9.82 | 7.83 | 8.09 | 5.66 | 4.46 | 7.60 | 6.91 | 9.09 | 4.12 |
| PHCA | 4.11 | 5.02 | 3.44 | 2.90 | 0.00 | 3.53 | 3.82 | 5.51 | 11.25 |
| PINK | 1.69 | 6.46 | 5.87 | 4.09 | 5.15 | 0.00 | 6.34 | 7.94 | 8.92 |

TABLE V
Values in mg/g of the HPLC identified compounds of group 1 (BRP1).

| | Samples | | | | | | | | | | |
|---|---|---|---|---|---|---|---|---|---|---|---|
| Abbrev. | 32 | 36 | 87 | 88 | 90 | 92 | 93 | 94 | 98 | 99 | 100 |
| CAF | 0.96 | 1.08 | 0.65 | 0.00 | 0.55 | 0.04 | 0.67 | 0.92 | 0.43 | 0.76 | 0.58 |
| CAF1 | 0.00 | 2.53 | 8.55 | 0.00 | 2.19 | 5.30 | 0.57 | 2.62 | 2.29 | 1.87 | 3.74 |
| CAF2 | 0.00 | 0.00 | 1.07 | 0.00 | 0.00 | 0.56 | 0.00 | 0.19 | 0.00 | 0.00 | 0.49 |
| CAF3 | 0.00 | 0.00 | 0.00 | 0.00 | 0.00 | 0.00 | 0.00 | 0.60 | 0.00 | 0.00 | 0.00 |
| CIN1 | 0.00 | 0.00 | 0.00 | 0.00 | 0.00 | 0.00 | 0.00 | 0.00 | 0.00 | 2.08 | 0.09 |
| DCBEN | 0.00 | 17.56 | 1.68 | 0.00 | 1.37 | 0.00 | 0.00 | 1.56 | 0.68 | 0.79 | 2.36 |
| DHCA | 12.30 | 16.95 | 39.24 | 37.99 | 22.58 | 29.46 | 23.56 | 29.89 | 19.50 | 23.15 | 27.17 |
| DHCA1 | 0.00 | 1.30 | 2.31 | 0.76 | 0.12 | 0.56 | 0.00 | 0.70 | 7.78 | 0.69 | 0.76 |
| DHCA2 | 0.00 | 1.35 | 6.46 | 0.85 | 0.25 | 1.04 | 0.00 | 0.78 | 1.30 | 0.71 | 0.68 |
| DHCA3 | 0.00 | 5.31 | 0.36 | 0.50 | 1.08 | 0.46 | 0.00 | 0.15 | 0.17 | 0.93 | 0.23 |
| DHCA4 | 0.00 | 0.00 | 0.00 | 1.45 | 0.65 | 0.00 | 0.00 | 0.56 | 0.69 | 0.60 | 0.51 |
| DHCA5 | 0.00 | 0.00 | 0.00 | 0.46 | 0.50 | 0.00 | 0.00 | 0.00 | 0.31 | 2.52 | 2.04 |
| DHCA6 | 0.00 | 0.00 | 0.00 | 0.47 | 2.29 | 0.00 | 0.00 | 0.00 | 0.50 | 2.05 | 0.23 |
| DHCA7 | 0.00 | 0.00 | 0.00 | 0.00 | 0.66 | 0.00 | 0.00 | 0.00 | 2.15 | 0.89 | 4.12 |
| DHCA8 | 0.00 | 0.00 | 0.00 | 0.00 | 4.12 | 0.00 | 0.00 | 0.00 | 0.00 | 0.00 | 0.00 |
| DPB | 6.17 | 16.09 | 8.68 | 21.64 | 17.49 | 0.00 | 5.35 | 8.88 | 29.69 | 12.45 | 15.36 |
| E | 36.30 | 54.87 | 88.84 | 26.35 | 18.79 | 71.29 | 40.45 | 47.81 | 54.69 | 36.31 | 59.55 |
| FER | 0.00 | 2.87 | 2.81 | 0.00 | 0.57 | 3.12 | 0.00 | 0.00 | 0.40 | 0.33 | 0.70 |
| KAEMP1 | 17.66 | 11.40 | 20.88 | 17.28 | 13.42 | 49.58 | 4.37 | 16.44 | 16.77 | 11.54 | 13.19 |
| P-CUM | 3.56 | 6.78 | 13.09 | 9.97 | 7.57 | 4.37 | 7.28 | 13.18 | 7.18 | 4.58 | 9.57 |
| PHCA | 4.47 | 7.09 | 6.61 | 18.52 | 14.40 | 0.00 | 5.55 | 8.39 | 11.40 | 10.01 | 9.63 |
| PINK | 8.31 | 5.88 | 17.54 | 0.00 | 7.10 | 7.04 | 6.95 | 12.48 | 22.12 | 9.42 | 11.19 |

DHCA from 15 to 40 mg/g ou 1.5 to 4.0%.
E from 40 to 90 mg/g ou 4.0 to 9.0%.
PCB from 15 to 40 mg/g or 1.5 to 4.0%.
P-CUM from 7 to 15 mg/g or 0.7 to 1.5%.
PHCA from 8 to 20 mg/g or 0.8 to 2.0%.
PINK from 8 to 25 mg/g or 0.8 to 2.5%.

TABLE VI
Values in mg/g of the HPLC identified compounds of group 1 (BRP2).

| | Samples | | | | | | | | |
|---|---|---|---|---|---|---|---|---|---|
| Abbrev. | 13 | 26 | 27 | 28 | 30 | 31 | 33 | 40 | 85 |
| CAF | 1.05 | 0.80 | 0.90 | 0.91 | 0.75 | 0.00 | 0.75 | 1.47 | 0.43 |
| CAF1 | 4.46 | 3.85 | 3.72 | 2.29 | 0.00 | 0.00 | 0.00 | 2.68 | 1.29 |
| CAF2 | 1.69 | 2.01 | 0.00 | 0.00 | 0.00 | 0.00 | 0.00 | 0.00 | 0.16 |
| DCBEN | 3.69 | 15.49 | 13.74 | 11.31 | 12.72 | 15.27 | 14.65 | 22.06 | 0.95 |
| DHCA | 12.46 | 14.71 | 16.06 | 11.36 | 13.40 | 10.67 | 14.77 | 11.74 | 16.43 |
| DHCA1 | 1.60 | 2.51 | 2.62 | 1.72 | 1.63 | 1.29 | 0.00 | 5.02 | 0.72 |
| DHCA2 | 1.61 | 4.79 | 0.00 | 0.00 | 3.75 | 0.00 | 0.00 | 5.29 | 0.33 |
| DHCA3 | 0.00 | 3.33 | 0.00 | 0.00 | 2.37 | 0.00 | 0.00 | 2.62 | 0.87 |
| DPB | 4.19 | 7.60 | 7.13 | 6.98 | 12.50 | 16.91 | 4.87 | 15.99 | 16.07 |
| E | 23.71 | 38.13 | 47.99 | 44.09 | 36.17 | 30.69 | 63.04 | 56.92 | 36.3 |
| FER | 2.81 | 2.53 | 2.29 | 0.00 | 0.00 | 0.00 | 0.00 | 3.12 | 0.34 |
| KAEMP1 | 18.11 | 14.70 | 18.66 | 0.00 | 0.00 | 0.00 | 0.00 | 0.00 | 12.26 |
| P-CUM | 9.82 | 7.83 | 8.09 | 5.66 | 4.46 | 7.60 | 6.91 | 9.09 | 4.12 |
| PHCA | 4.11 | 5.02 | 3.44 | 2.90 | 0.00 | 3.53 | 3.82 | 5.51 | 11.25 |
| PINK | 1.69 | 6.46 | 5.87 | 4.09 | 5.15 | 0.00 | 6.34 | 7.94 | 8.92 |

DHCA from 10 to 15 mg/g or 1.0 to 1.5%.
E form 20 to 60 mg/g or 2.0 to 6.0%.
PCB from 4 to 15 mg/g or 0.4 to 1.7%.
P-CUM from 3 to 9 mg/g or 0.4 to 1.0%.
PHCA from 2 to 8 mg/g or 0.2 to 0.8%.
PINK from 2 to 8 mg/g or 0.2 to 0.8%.

TABLE VII
Values in mg/g of the HPLC identified compounds of group 1 (BRP3).

| | Samples | | | | | | | | | | | | |
|---|---|---|---|---|---|---|---|---|---|---|---|---|---|
| Abbrev. | 1 | 2 | 5 | 6 | 7 | 9 | 12 | 14 | 15 | 16 | 18 | 96 | 97 |
| CAF | 0.00 | 0.00 | 0.00 | 0.00 | 0.00 | 0.34 | 0.50 | 0.54 | 1.03 | 0.86 | 0.48 | 0.31 | 0.41 |
| CAF1 | 0.64 | 0.00 | 1.11 | 0.00 | 1.55 | 0.00 | 1.24 | 1.85 | 1.03 | 2.21 | 0.00 | 2.72 | 2.43 |
| CAF2 | 1.81 | 0.00 | 0.00 | 0.00 | 0.00 | 0.00 | 0.00 | 0.57 | 3.58 | 1.31 | 0.00 | 0.18 | 0.16 |
| DCBEN | 4.48 | 1.18 | 0.00 | 1.83 | 0.00 | 0.00 | 0.00 | 2.16 | 2.24 | 2.90 | 4.38 | 0.30 | 0.29 |
| DHCA | 2.44 | 1.57 | 7.87 | 8.18 | 7.40 | 6.92 | 7.20 | 10.58 | 7.09 | 6.45 | 5.75 | 5.38 | 6.73 |
| DHCA1 | 0.00 | 0.75 | 0.88 | 3.40 | 3.17 | 1.87 | 0.46 | 0.93 | 0.69 | 0.83 | 0.58 | 0.18 | 0.22 |
| DHCA2 | 0.00 | 2.30 | 2.28 | 1.08 | 0.73 | 0.76 | 0.48 | 0.88 | 1.25 | 0.88 | 2.04 | 0.66 | 0.71 |
| DHCA3 | 0.00 | 0.00 | 0.71 | 1.27 | 3.02 | 0.87 | 0.70 | 1.00 | 0.00 | 1.71 | 1.57 | 0.20 | 0.20 |
| DHCA4 | 0.00 | 0.00 | 0.68 | 0.00 | 1.42 | 2.54 | 0.89 | 0.95 | 0.00 | 0.00 | 0.53 | 0.63 | 0.77 |
| DHCA5 | 0.00 | 0.00 | 1.01 | 0.00 | 1.05 | 0.00 | 1.35 | 0.80 | 0.00 | 0.00 | 1.71 | 0.11 | 0.61 |
| DHCA6 | 0.00 | 0.00 | 0.00 | 0.00 | 0.00 | 0.00 | 0.00 | 0.00 | 0.00 | 0.00 | 0.00 | 0.51 | 0.00 |
| DPB | 4.60 | 1.51 | 8.60 | 4.41 | 5.85 | 4.26 | 5.84 | 4.08 | 2.05 | 10.06 | 6.71 | 8.05 | 9.62 |
| E | 13.79 | 4.63 | 9.77 | 17.85 | 21.27 | 12.73 | 12.91 | 21.50 | 17.76 | 10.87 | 11.75 | 11.68 | 13.59 |
| FER | 0.00 | 0.00 | 0.00 | 0.00 | 1.84 | 7.34 | 0.83 | 0.98 | 2.20 | 1.69 | 0.24 | 0.66 | 0.47 |
| H | 0.00 | 0.00 | 0.00 | 0.00 | 0.00 | 0.00 | 0.00 | 0.00 | 0.00 | 0.00 | 0.00 | 0.00 | 0.00 |
| KAEMP1 | 0.00 | 0.00 | 10.66 | 12.35 | 10.64 | 0.00 | 10.93 | 13.97 | 0.00 | 0.00 | 3.83 | 5.24 | 1.59 |
| P-CUM | 0.00 | 0.00 | 0.26 | 3.08 | 3.98 | 2.47 | 2.58 | 5.39 | 5.66 | 2.33 | 0.50 | 2.98 | 2.40 |
| PHCA | 0.00 | 0.00 | 0.00 | 0.00 | 0.00 | 0.00 | 2.25 | 2.54 | 2.05 | 3.30 | 1.27 | 3.41 | 4.07 |
| PINK | 0.00 | 0.00 | 0.00 | 3.38 | 6.52 | 5.51 | 3.86 | 0.57 | 3.58 | 1.31 | 1.48 | 5.15 | 5.67 |

DHCA from 1 to 10 mg/g or 0.1 to 1.0%.
E from 2 to 20 mg/g or 0.2 to 2.0%.
PCB from 1.0 to 10 mg/g or 0.1 to 1.0%.
P-CUM from 0.25 to 5 mg/g or 0.025 to 0.5%.
PHCA from 0.25 to 4 mg/g or 0.025 to 0.4%.
PINK from 0.1 to 5 mg/g or 0.01 to 0.5%.

TABLE XVI
Values in mg/g of HPLC identified compounds of group 2 (BRG)*.

| | Samples | | | | | | | | |
|---|---|---|---|---|---|---|---|---|---|
| Abbrev. | 55 | 60 | 61 | 62 | 66 | 68 | 75 | 82 | 89 |
| DCBEN | 0.00 | 0.00 | 0.00 | 0.01 | 0.00 | 0.00 | 0.00 | 0.03 | 0.00 |
| I | 0.00 | 0.00 | 0.00 | 0.02 | 0.05 | 0.00 | 0.00 | 0.00 | 0.00 |
| G2 | 0.00 | 0.00 | 0.01 | 0.01 | 0.03 | 0.00 | 0.09 | 0.04 | 0.96 |
| DHCA | 0.00 | 0.06 | 0.00 | 0.00 | 0.00 | 0.00 | 0.00 | 0.07 | 0.00 |
| DPB | 0.00 | 0.08 | 0.67 | 0.02 | 0.00 | 0.00 | 0.11 | 0.13 | 0.00 |
| FER | 0.00 | 0.00 | 0.00 | 0.00 | 0.00 | 0.00 | 0.05 | 0.02 | 0.00 |
| P-CUM | 0.00 | 0.02 | 0.01 | 0.00 | 0.00 | 0.00 | 0.03 | 0.00 | 0.00 |
| E | 0.00 | 0.07 | 0.29 | 0.02 | 0.00 | 0.00 | 0.27 | 0.19 | 0.00 |
| H | 0.00 | 0.00 | 0.10 | 0.00 | 0.12 | 0.16 | 6.10 | 2.04 | 0.00 |
| K | 0.00 | 0.00 | 0.00 | 0.00 | 0.00 | 0.00 | 0.00 | 0.00 | 0.00 |
| L1 | 0.00 | 0.00 | 0.06 | 0.14 | 0.00 | 0.00 | 0.00 | 0.00 | 2.56 |
| L2 | 0.00 | 0.00 | 0.00 | 0.00 | 0.07 | 0.00 | 0.00 | 0.00 | 0.00 |
| M | 0.00 | 0.00 | 0.00 | 0.18 | 0.03 | 0.00 | 0.00 | 0.00 | 0.00 |
| N | 0.00 | 0.00 | 0.00 | 0.00 | 0.00 | 0.00 | 0.00 | 0.00 | 0.00 |
| KAEMP1 | 0.00 | 0.04 | 0.00 | 0.00 | 0.00 | 0.00 | 0.00 | 0.00 | 0.00 |
| PINK | 0.00 | 0.00 | 0.00 | 0.00 | 0.00 | 0.00 | 0.00 | 0.00 | 0.00 |
| G1 | 0.08 | 0.02 | 0.05 | 0.08 | 0.15 | 0.01 | 0.26 | 0.14 | 7.69 |

*This group contains few samples, having no conditions be statistically analysed by the multivariation method.

TABLE XVII
Biological properties of Brazilian propolis compounds

| | Biological Activities | | | | | | |
|---|---|---|---|---|---|---|---|
| | | | Hepato- | | | | |
| | | | protection | | | | |
| | Antitumor activity (LD₅₀ μg/mL) | | Effect | MIC (H. pylori mg/mL) | | | Antioxidant |
| Compound | HT1080 | | IC₅₀ μM | NTCT | NTCT | ATCC | Activity |
| Number | (Fibrosarcoma) | L5-26 (Colon) | (Hepatocytes) | 11637 | 11916 | 43504 | (DPPH) (μg/mL) |
| 1 | 71.53 | 77.07 | — | 0.13 | 0.25 | 0.25 | — |
| 2 | 46.86 | 50.22 | 88.1 | 0.50 | 0.50 | 1.00 | — |
| 3 | >100 | >100 | — | >1 | >1 | >1 | — |
| 4 | >100 | >100 | — | — | — | — | — |
| 5* | 45.47 | 59.32 | — | >1 | >1 | >1 | 5.6 |
| 6 | 25.94 | 77.90 | 26.9 | 0.25 | 0.50 | 0.50 | — |
| 7 | >100 | >100 | 34.6 | >1 | >1 | >1 | — |
| 8 | >100 | >100 | 22.1 | >1 | >1 | >1 | — |
| 9 | 4.05 | 10.44 | 45.8 | >1 | >1 | >1 | 11.1 |
| 10 | 72.91 | 63.54 | 94.4 | 0.50 | 0.50 | 1.0 | — |
| 11 | 70.10 | 73.27 | 80.2 | 0.13 | 0.25 | 0.25 | — |
| 12 | 75.43 | 95.92 | 79.6 | >1 | >1 | >1 | — |
| 13 | 72.82 | >100 | — | 0.13 | 0.13 | 0.13 | — |
| 14 | 75.43 | 95.92 | — | >1 | >1 | >1 | — |
| 15 | 94.86 | >100 | 44.1 | >1 | >1 | >1 | — |
| 16 | 57.40 | 58.09 | — | — | — | — | — |
| 17 | 45.48 | 57.15 | — | >1 | >1 | >1 | — |
| 18 | >100 | >100 | — | 1.0 | 1.0 | 1.0 | — |
| 19 | 5.83 | 4.85 | 12.7 | >1 | >1 | >1 | — |
| 20 | 2.91 | 5.95 | 17.6 | >1 | >1 | >1 | 4.8 |
| 21 | 2.30 | 7.64 | 15.0 | 0.50 | 1.0 | >1 | — |
| 22 | 26.97 | 70.98 | 22.0 | 1.0 | 1.0 | >1 | — |
| 23 | 38.92 | 39.86 | 47.2 | >1 | >1 | >1 | 61.9 |
| 24 | >100 | >100 | 15.2 | 0.13 | 0.25 | 0.25 | 12.2 |
| 25 | >100 | >100 | — | 0.25 | 0.13 | 0.13 | 15.0 |
| 26 | 13.9 | 12.4 | 63.5 | >1 | >1 | >1 | 29.1 |
| 27 | 43.2 | 13.7 | 15.0 | >1 | >1 | >1 | 30.1 |

| | | | | Antitumor activity | | |
|---|---|---|---|---|---|---|
| | | | | (IC₅₀ μM) | | |
| | | | | B16-BL6 | A-549 (lung | |
| | | | | (melanoma) | carcinoma) | |
| 28* | >195 μM | 44.5 μM | 78 | >195 | >195 | — |
| 29 | 116 μM | 37.7 μM | — | 127 | >190 | — |
| 30 | 157 μM | 76.3 μM | — | 105 | >190 | — |
| 31 | 119 μM | 52.4 μM | — | 122 | >190 | |
| 32 | — | — | — | — | — | — |
| 33 | 337 μM | 111 μM | — | 314 | 387 | — |
| 34 | — | — | — | — | — | — |
| 35 | — | — | — | — | — | — |

*Compound 5 has been evaluated in other tumor lines.
LD₅₀ values expressed in μg/mL:
NCI460 (lungs tumor): 9.0;
UACC62 (melanoma): 6.5;
MCF7 (normal breast tumor): 11.0;
NCIADR (breast tumor multiple drugs resistance phenotype): 23.0 (M. C. Marcucci and J. E. Carvalho. Not published results).

TABLE XVIII
Brazilian propolis compounds

| Compound | Structure |
|---|---|
| 3-Hydroxy-2,2-di- methyl-8-prenyl-2H-1-benzo- pyran-6-propenoic acid \mathring{1} | 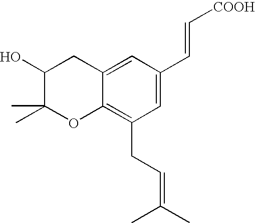 |
| 2,2-Dimethyl-8-prenyl-2H-1-benzo- pyran-6-propenoic acid (DPB) \mathring{2} | 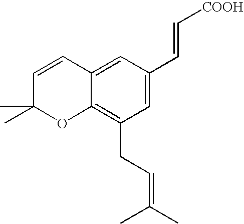 |
| 2,2-Dimethyl-2H-1-benzo- pyran-6-propenoic acid \mathring{3} | 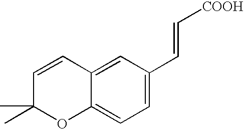 |
| 2,2-Dimethyl-2H-1-benzo- pyran-6-carboxylic acid \mathring{4} | 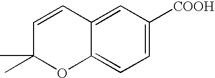 |
| 3,5-Diprenyl-4-hydroxy- cinnamic acid (DHCA) \mathring{5} | 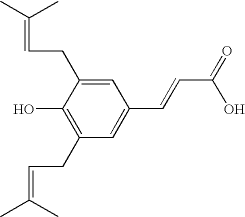 |
| 4-Dihydroxicinnamoyl-oxy-3-prenyl- cinnamic acid \mathring{6} | 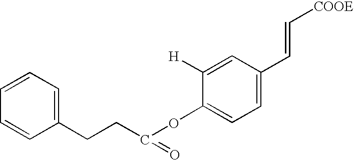 |
| 3-Prenyl-4-hydroxy- cinnamic acid (PHCA) \mathring{7} | 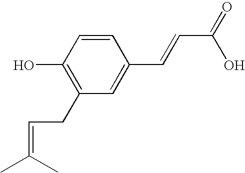 |
| Vanillin (G1) \mathring{8} | 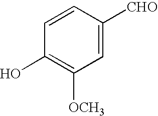 |
| 3-Methoxy-4-hydroxy- cinnamaldehyde (compound G2 - coniferyl aldehyde) \mathring{9} | 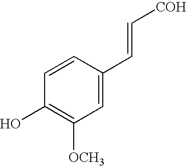 |
| Isocupressic acid \mathring{10} | 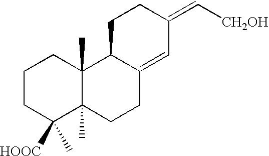 |
| 15-Acetoxyisocupressic acid 11 | 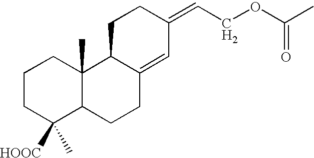 |
| Agathic acid 12 | 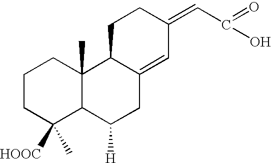 |
| Agathic acid 15-methyl ester 13 | 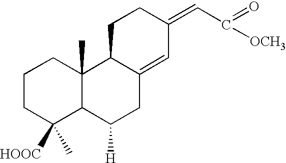 |
| Agathalic acid 14 | 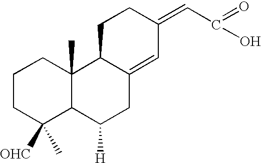 |
| Cupressic acid 15 | 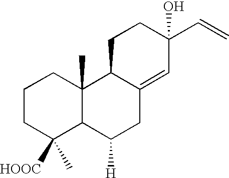 |
| Tremetone 16 | 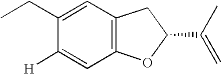 |
| Viscidone 2-[1-Hydroxy- methyl]vinyl-5-ace- thyl-hydroxy- cumarane (I) 17 | 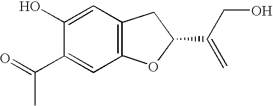 |
| 12-Acethylviscidone 18 | 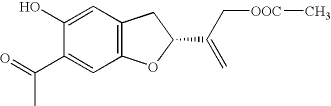 |
| Betuletol (flavonol) 19 | 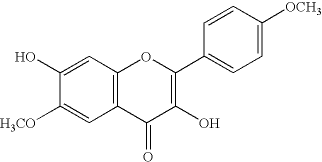 |
| Kaempferide (flavonol) 20 | 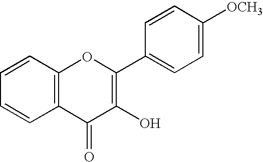 |
| Ermanin (Flavonoid) 21 | 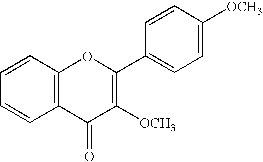 |
| 3,5,7-Trihydroxy-4′-methoxy- flavonol (Flavonol) 22 | 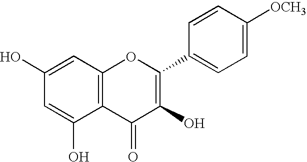 |
| Dimeric coniferyl acetate 23 | 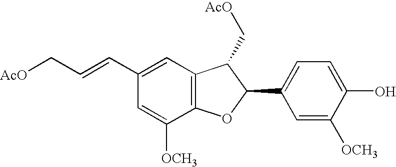 |
| (E)-3-{4-hydroxy-3-[(E)-4-(2,3-di- hydrocinnamoyloxy)-3-meth- yl-2-butenyl]-5-pre- nyl}-2-propenoic acid 24 | 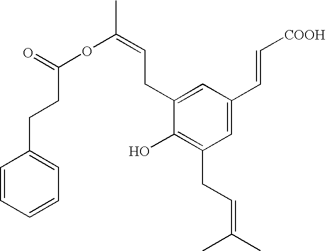 |
| (E)-3-[2,3-Dihydro-2-(1-methyl- ethenyl)-7-pre- nyl-5-benzofuranyl]-2-propenoic acid 25 | 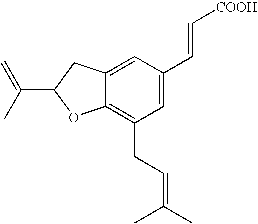 |
| Benzofuran A 26 | 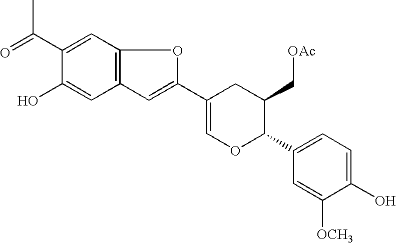 |
| Benzofuran B 27 | 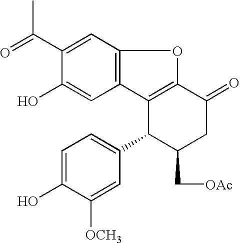 |
| 3,4-Di-O-caffeoylquinic acid 28 | 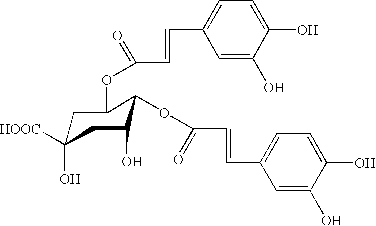 |
| Metyl-3,4-di-O-caffeoylquinic acid 29 | 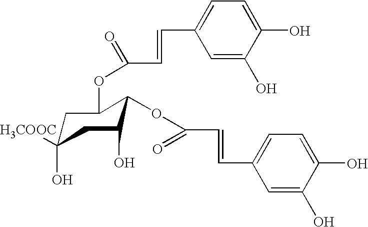 |
| Methyl-4,5-di-O-caffeoylquinic acid 30 | 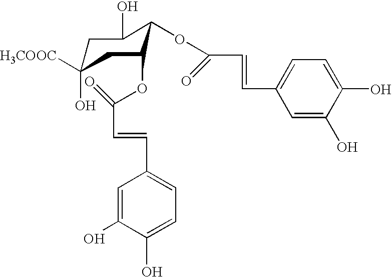 |
| 3,5-Di-O-caffeoylquinic acid 31 | 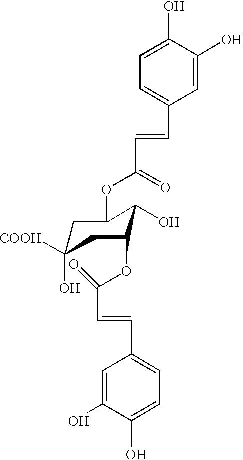 |
| 3-O-Caffeoylquinic acid 32 | 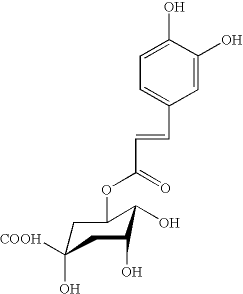 |
| Caffeic acid 33 | 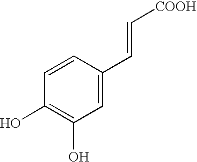 |
| Ferulic acid 34 | 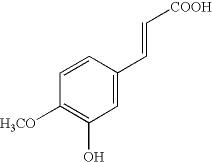 |
| p-Coumaric acid 35 | 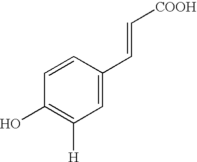 |

1) PROCESS FOR TYPING NATURAL PRODUCTS, characterized by comprising:extracting the raw material collected by honeycomb scraping; micro scale dissolving the raw material in hot methanol, paper filtering and millipore filtering for further anaysis; only applying one dilution factor for raw state samples; employing one chromatographic column for complex sample separation; running analysis in liquid chromatograph having a photodiode lattice and an automatic injector, into a maximum analysis time of 50 minutes and the detection being effected at 280 and 340 nm wavelenghts; co-chromatography of the reference compounds and the experimental sample; employing a suitable software for treating the obtained results; united applying the gas chromatography and mass spectrometry techniques:

2) PROCESS, according to claim 1, characterized in that the micro scale amount of the collected material is ranging from 50 and 150 mg and is solubilized in a volume of methanol ranging from 1.0 and 5,0 mL.

3) PROCESS, according claim 1, characterized in that the dilution factor comprises from 1 to 10% of dilution in relation to the original extract.

4) PROCESS, according to claim 1, characterized in that the analysis are run in a liquid chromatograph having a photodiode lattice and an automatic injector; the chromatographic conditions comprise a movable phase of water-formic acid and methanol (chromatographic grade) and the eluition is run at a flow of 1 mL/min by means of linear gradient.

5) PROCESS, according to claim 1, characterized in that the maximum analysis time comprises 50 minutes and the detection is effected at 280 e 340 nm wavelenghts.

6) PROCESS, according to claim 1, characterized in that the reference compounds are co-chromatography performed, joined the experimental sample.

7) PROCESS, according to claim 1,, characterized in that a suitable software is employed for treating the obtained results.

8) PROCESS, according to claim 1, characterized in that process also comprises gas chromatography analysis combined to a mass spectrometry analysis.

9) PROCESS, according to claim 8, characterized in that comprises analysis of 5-10 mg of dry própolis methanolic extract solubilized in 10-20 μL of pyridine and after 100 μL of reactive bis-(trimethylsilil)trifluoroacetamide (BSTFA) containing 1% of trimethyilchlorinesilane (TMCS).

10) PROCESS, according to claim 9, characterized in that the mixture for analysis is incubated at 100° C. for 30 minutes in a closed tube.

11) PROCESS, acording to claim 8, characterized in that the derived and separated are analysed in a capilar column (at 5% of phenylmethylsilicone) 30 m, 0.25 mm inner diameter, He carrier gas, solvent delay of 4 minutes, mass detector temperature corresponding to 280° C., injector temperature corresponding to 250° C., range of analysed mass comprising 40-650 a.m.u, flow corresponding to 1 mL/min, split relation: 1/100, operation pressure 0.73 bar (100° C.), t mperature biasing at 100° C. (initial), for 2 min, velocity: 8° C./min up to 200° C. for 2 min at 5° C./min up to 310° C. for 6 min; separation time corresponding to 40 minutes.

12) PROCESS, according to claim 1, , characterized in that after injectio in the chromatograph, the Brazilian propolis is identified by means of a marker.

13) PROCESS, according to claim 12, characterized in that the marker is a majority component of the analysed samples.

14) PROCESS, according to claim 13, characterized in that the marker comprises the 3,5-diprenyl-4-hydroxycinnamic acid (DHCA) which divides, by typing, the Brazilian propolis into two groups.

15) PROCESS, according to claim 14, characterized in that the two types of propolis are divided into two subdividions.

16) PROCESS, according claim 15, , characterized in that one type of propolis is subdvided based on the decreased content of the DHCA marker in the sample.

17) PROCESS, according to claim 16, characterized in that one of subdivisions comprises the BRP 1 group with high content of markers: DHCA (from 15 to 40 mg/g or 1.5 to 4.0%) E (from 40 to 90 mg/g or 4.0 to 9.0%) DPB (from 15 to 40 mg/g or 1.5 to 4.0%) P-CUM (from 7 to 15 mg/g or 0.7 to 1.5%) PHCA (from 8 to 20 mg/g or 0.8 to 2.0%) PINK (from 8 to 25 mg/g or 0.8 to 2.5%).

18) PROCESS, according to claim 16, characterized in that the second subdivision comprises the BRP 2 group having intermediary marker concentration: DHCA (from 10 to 15 mg/g or 1.0 to 1.5%) E (from 20 to 60 mg/g or 2.0 to 6.0%) DPB (from 4 to 15 mg/g or 0.4 to 1.7%) P-CUM (from 3 to 9 mg/g or 0.4 to 1.0%) PHCA (from 2 to 8 mg/g or 0.2 to 0.8%) PINK (from 2 to 8 mg/g or 0.2 to 0.8%).

19) PROCESS, according to claim 16, characterized in that the third subdivision comprises the BRP 3 group: Low markers concentration: DHCA (from 1 to 10 mg/g or 0.1 to 1.0%) E (from 2 to 20 mg/g or 0.2 to 2.0%) DPB (from 1.0 to 10 mg/g or 0.1 to 1.0%) P-CUM (from 0.25 to 5 mg/g or 0.025 to 0.5%) PHCA (from 0.25 to 4 mg/g ou 0.025 a 0.4%) PINK (de 0.1 a 5 mg/g ou 0.01 a 0.5%).

20) PROCESS, according to claim 15, characterized in that another type of propolis is subdivided based on the decreasing content of the submarker combined to other present substances.

21) PROCESS, according to claim 20, characterized in that the submarker is the G1 compound and the other substances are PHCA, DCBEN and DPB.

22) PROCESS, according to claim 20, characterized in that the first subdivision comprises the BRG 1 which has na intermediary concentration of the submarker G₁ and low concentration of the DHCA marker and compounds PHCA, DCBEN and DPB.

23) PROCESS, according to claim 20, characterized in that the second subdivision comprises the BRG 2 having intermediary-to-low concentration of a submarker G₁ and low concentration or absence of the DHCA marker and the PHCA, DCBEN and DPB. compounds

24) PROCESS, according to claim 20, characterized in that the third subdivision comprises the BRG 3 having low concentration of submarker G₁ and low concentration or absence of the DHCA marker and PHCA, DCBEN and DPB compounds.

25) PROCESS, according to claim 1, characterized in that the software is the HPLC-propolis system, comprising the software application suitable to a Windows type operational system, which is employed as an analysis tool for data treatment of the chromatograph results.

26) PROCESS, according to claim 25, characterized in that the software data input is made by means of a file generated from the chromatograph containing information provided by the user and information previous recorded in the software.

27) PROCESS, according to 26, characterized in that the file generated by the chromatograph is software read and the data are inserted in a data bank; the file information refers to the compounds of the sample, and comprises information related to compound code, column retention time and compound chromatograph area.

28) PROCESS, according to claim 27, characterized in that typed information comprises user name, user password, name or number of the chromatograph result, name or number of the sample, geographic origin, botanical origin, collection date, physical state of the sample, injection volume of the sample in the chromatograph, and sample dilution grade.

29) PROCESS, according to claim 28, characterized in that the software previous recorded information comprises compound code, name of compound, column retention time, and compound chromatograph area; all data referring to the standard reference.

30) PROCESS, according to claim 29, characterized in that the information processing comprises effect concentration calculation of each separated compound, chromatography result of proplolis sample; the calculation considers the chromatogram area peak in relation to the known standard reference area; are also considered the dilution factor, the chromatogram peak concentration result is reinserted in the software data bank for further consultation; these data informs about compound quality, are displayed in an output Table and finally the typing is defined for each sample, depending on the concentration of principal sample components.

31) PROCESS, according to claim 30, characterized in that the information output is made by a software generated report; the report contains: chromatogram name or number, sample name or number, compound code, compound name, column retention time, the obtained concentration of the previous processing and the sample group definition (type); this report can be screend at computer video or paper printed, data analysis system permits new standard reference data insertion, since the standard references are insulated and identified in propolis sample; This system also permits quantification and determination of any natural product by means of HPLC and/or GC-MS, since the present active component chemical structure is known.
